(19) United States
(12) Patent Application Publication
(10) Pub. No.: US 2009/0130073 A1
(43) Pub. Date: May 21, 2009

(54) Microorganisms Inhibiting the Formation of Foot Malodor

(75) Inventors: Andreas Reindl, Mannheim (DE); Rolf Knoll, Laudenbach (DE); Christine Lang, Berlin (DE); Markus Veen, Berlin (DE); Eckhard Budde, Berlin (DE); Mewes Boettner, Berlin (DE)

Correspondence Address: CONNOLLY BOVE LODGE & HUTZ, LLP, P O BOX 2207, WILMINGTON, DE, 19899, US

(73) Assignee: BASF SE, Ludwigshafen (DE)

(21) Appl. No.: 12/067,229

(22) PCT Filed: Sep. 18, 2006
(86) PCT No.: PCT/EP2006/009069

§ 371 Date: Mar. 18, 2008

Related U.S. Application Data
(60) Provisional application No. 60/740,224, filed on Nov. 28, 2005.

(30) Foreign Application Priority Data
Sep. 19, 2005 (EP) 05020391.8
Sep. 22, 2005 (EP) 05020706.7
Nov. 21, 2005 (EP) 05025354.1

(51) Int. Cl. A61K 35/74; C12N 1/20

(52) U.S. Cl. 424/93.45; 435/252.1; 435/252.9; 424/93.4

(57) ABSTRACT

Described are microorganisms which are able to inhibit the formation of foot malodor by skin microorganisms. Also described are compositions comprising such microorganisms as well as the use of such microorganisms in cosmetic, prophylactic or therapeutic applications.

The present invention relates to microorganisms which are able to prevent the generation of foot malodor by skin microorganisms. The present invention also relates to compositions, comprising such microorganisms, e.g. cosmetic or pharmaceutical compositions and to the use of such micoorganisms in cosmetic, prophylactic or therapeutic applications.

It is well known in the art that offensive body odors are generated by the decomposition of organic substances derived from sweat glands or skin constituents into short-chain fatty acids (Kanda et al. Br. J. Dermat. 122 (1990), 771-776). In particular, foot malodor develops as feet are enclosed inside footwear and sweat. Feet have a higher number of sweat glands than other parts of the body, so that they can sweat excessively and the produced sweat cannot evaporate due to the enclosure of the feet in footwear. Microorganisms which live as commensales on the surface of the foot skin and which are able to metabolize the sweat components or skin constituents cause the malodor. It is a well accepted fact in the art that the key odorant of malodorant feet is isovaleric acid. Kanda et al. (Br. J. Dermat. 122 (1990), 771-776), for instance, found that short fatty acids, in particular isovaleric acid are generated in greater amounts by people with strong foot odor than by persons with no apparent foot odor. Isovaleric acid was present in all subjects with foot odor but was not detectable in those without and accumulated in socks and shoes, leading to the generally known smell of foot textiles.

Isovaleric acid is mainly generated by the enzymatic conversion of the branched amino acid leucine. A comparable enzymatic process is already known from the production of Swiss cheese, where microorganisms are purposely used to generate isovaleric acid as a major flavor constituent (Thierry et al., Appl. Env. Microbiol. 68(2) (2002), 608-615). Dairy propionic acid bacteria constitute one of the major floras that grow during the ripening of Swiss type cheese and are involved in the formation of the characteristic flavor. Thierry et al. (Appl. Env. Microbiol. 68(2) (2002), 608-615) could show that Propionibacterium freudenreichii is capable of degrading branched amino acids and that the transamination of leucin by said bacterium produced α-ketoisocaproic acid which was subsequently enzymatically converted to isovaleric acid. Specific foot microorganisms which are involved in the biosynthesis of isovaleric acid have not yet been described in the prior art.

Most of the currently available products on the market which aim at reducing or eliminating offensive body odors, contain either strong perfumes to mask body odor or aluminium salts to inhibit perspiration or rely on an unspecific reduction of the biological activity of foot microorganisms, i.e. the eradication of the bacteria responsible for causing the odor. In fact, cosmetic deodorants generally contain antibacterial compounds which inhibit the growth of the skin micro flora, like, for instance, 2,4,4′-trichloro-2′hydroxy-diphenyl-ether (Triclosan). However, while this principle is effective against odor formation, it leads to a severe destruction of the natural residential microbial skin flora that protects the skin, e.g. from being colonized by potentially pathogenic microorganisms (Bisno et al., Am. J. Med. 76 (5A) (1984), 172-179).

Thus, there is a need for means and methods allowing to inhibit the formation of foot malodor without causing severe side-effects or destructing the microbial skin flora of the human foot.

The present invention addresses this need and provides microorganisms and methods which lead to the prevention of foot malodor by skin microorganisms. In particular, it provides the embodiments as characterized in the claims.

Accordingly, the present invention in a first aspect relates to a microorganism which is able to prevent the generation of foot malodour by skin microorganisms.

The inventors surprisingly found that an effective prevention of the generation of foot malodour can be achieved by administering to the skin the above described microorganisms or inactivated forms thereof. The inventors for the first time identified corresponding microorganisms and provided methods for their identification. These microorganisms are able to inhibit the formation of isovaleric acid by odor generating microorganisms from the skin or to inhibit the growth of foot malodour generating skin microorganisms. By this, the generation of typical foot smell is suppressed.

The term “foot malodor” relates to a typical foot odor which is generally described as cheesy. The “foot malodor” is generally generated by skin microorganism which are residing in the skin regions of the foot. Preferably the term means that a typical foot odor can be detected. More preferably, the term means that the detection of the typical foot odor is verified by sniffing with the nose, preferably the nose of a skilled person. For the purpose of the present invention preferably different categories of foot odor intensity are used. Preferably these categories may be defined as: 0 (no odor detectable), 1 (slight odor detectable), 2 (odor detectable) and 3 (strong odor detectable). The term “prevent” in connection with the generation of foot malodor means that the release of the typical cheesy foot odor is stopped or decreased when the skin is contacted with a microorganism according to the invention. Preferably, the term relates to a lowering of any of the herein above described categories of foot odor intensity from any higher number to any lower number. A “stopped release” means the cheesy foot odor is not detectable when a microorganism according to the invention is contacted with the skin. Preferably, the term means that the category of the foot odor when a microorganism according to the invention is contacted with the skin is 0 (no odor detectable). A “decreased release” means that the cheesy foot odor is reduced when a microorganism according to the invention is contacted with the skin. The term “reduced” in connection with the release of the typical cheesy foot odor means that the odor intensity of skin contacted with a microorganism according to the invention is 2 (odor detectable), preferably 1 (slight odor detectable), and more preferably 0 (no odor detectable), if the odor intensity of skin not contacted with a microorganism according to the invention is 3 (strong odor detectable), or the odor intensity of skin contacted with a microorganism according to the invention is 1 (slight odor detectable) and preferably 0 (no odor detectable), if the odor intensity of skin not contacted with a microorganism according to the invention is 2 (odor detectable), or the odor intensity of skin contacted with a microorganism according to the invention is 0 (no odor detectable), if the odor intensity of skin not contacted with a microorganism according to the invention is 1 (slight odor detectable).

The detection by “sniffing with the nose” relates to a detection of typical foot malodor carried out by one or more persons having been trained or having not been trained for the detection of odor with their noses. The detection may be carried out in any suitable form or by using any suitable technique known to the person skilled in the art. Preferably, the detection may be carried out by a qualified panel of persons having been trained for the detection of foot malodor with their noses, more preferably it may be carried out by three persons which form a qualified panel. For the purpose of the present invention preferably different categories of odor intensity are used. Preferably these categories may be defined as: 0 (no odor detectable), 1 (slight odor detectable), 2 (odor detectable) and 3 (strong odor detectable). The person or persons forming the qualified panel may independently assess the odor intensity of odorous samples. The value of odor perception of the person(s) belonging to the qualified panel may be calculated by any means known to the person skilled in the art. Preferably, the mean value of odor perception of all person(s) belonging to the qualified panel may be calculated. Based on these data the intensity of odor may subsequently be quantified by any means known to the person skilled in the art.

In another preferred embodiment the detection of foot malodor may be carried out by a panel of persons as described in Kanda et al. (Br. J. Dermat. 122 (1990), 771-776). Moreover, the sniffing assay may be carried out as described in this document.

In a preferred embodiment the microorgansim which is able to prevent the generation of foot malodour by skin microorganisms is able to suppress the biosynthesis of isovaleric acid by skin microorganisms.

The term “biosynthesis of isovaleric acid” relates to the conversion of a precursor which can normally be found in foot sweat secret or which can result from dead skin cells or (dead) skin components into isovaleric acid, e.g. after having been decomposed into amino acids.

In particular, the term “precursor” relates to a chemical compound which is chemically or enzymatically converted in a reaction which leads to the production of isovaleric acid. Preferably, the term “precursor” relates to a branched-chain amino acid (BCAA) or derivative thereof. More preferably, the term relates to leucine or an amino acceptor like, for example, α-ketoglutarate or oxoglutarate. In a preferred embodiment leucin and α-ketoglutarate or oxoglutarate are used simultaneously in the form of a substrate and a co-substrate. In a further preferred embodiment the microorganism according to the invention suppresses the biosynthesis of at least one other compound which is odorous in the sense that it has a smell which resembles a typical foot odor. Examples for such compounds are 3-methylbutanal, 2-methylbutanal, ethyl ester of 3-methylbutanoic acid (isovaleric acid), in particular also compounds which are structurally related to isovaleric acid and which therefore could be considered to be derivatives thereof.

The term “odor”, “odorous” or “odoriferous” means that a typical foot odor can be detected. Preferably, the term means that the detection of the typical foot odor is verified by sniffing with the nose, preferably the nose of a skilled person. More preferably, the term refers to the amount of isovaleric acid or derivatives thereof which can be detected by GC/MS analysis. The term “odorless” means that a typical foot odor cannot be detected by sniffing with the nose, preferably the nose of a skilled person. More preferably, the term means that no isovaleric acid can be detected by GC/MS analysis. The verification by “sniffing with the nose” relates to a detection of typical foot malodor carried out by one or more persons having been trained or having not been trained for the detection of odor with their noses. More preferably, the term relates to a detection of isovaleric acid carried out by one or more persons having been trained or having not been trained for the detection of odor with their noses. The detection may be carried out in any suitable form or by using any suitable technique known to the person skilled in the art. Preferably the detection may be carried out by a qualified panel of persons having been trained for the detection of foot odor with their noses, more preferably it may be carried out by three persons which form a qualified panel. For the purpose of the present invention preferably different categories of odor intensity are used: 0 (no odor detectable), 1 (slight odor detectable), 2 (odor detectable) and 3 (strong odor detectable). The person or persons forming the qualified panel may independently assess the odor intensity of odorous samples of microorganisms. Preferably the odor of in vitro generated samples consisting of microorganisms, able to generate isovaleric acid from its precursor form, a precursor and a microorganism able to suppress the biosynthesis of isovaleric acid or corresponding control samples without microorganisms defined in the invention may be assessed. The value of odor perception of the person(s) belonging to the qualified panel may be calculated by any means known to the person skilled in the art. Preferably, the mean value of odor perception of all person(s) belonging to the qualified panel may be calculated. Based on these data the intensity of odor may subsequently be quantified by any means known to the person skilled in the art.

The term “skin” refers to the body's outer covering, as known to the person skilled in the art. Preferably the term relates to three layers: epidermis, dermis, and subcutaneous fatty tissue. The epidermis is the outermost layer of the skin. It typically forms the waterproof, protective wrap over the body's surface and is made up of stratified squamous epithelium with an underlying basal lamina. It usually contains no blood vessels, and is nourished by diffusion from the dermis. The main type of cells which make up the epidermis are keratinocytes, with melanocytes and Langerhans cells also present. The epidermis is divided into several layers where cells are formed through mitosis at the innermost layers. They move up the strata changing shape and composition as they differentiate and become filled with keratin. They eventually reach the top layer called stratum corneum and become sloughed off, or desquamated. The outermost layer of the epidermis consists of 25 to 30 layers of dead cells. Conventionally, the epidermis is divided into 5 sublayers or strata (from superficial to deep): the stratum corneum, the stratum lucidum, the stratum granulosum, the stratum spinosum and the stratum germinativum or stratum basale. Typically, the interface between the epidermis and dermis is irregular and consists of a succession of papillae, or fingerlike projections, which are smallest where the skin is thin and longest in the skin of the palms and soles. Typically, the papillae of the palms and soles are associated with elevations of the epidermis, which produce ridges. Subcutaneous fatty tissue is the deepest layer of the skin. A characteristic of this layer is that it is composed of connective tissue, blood vessels, and fat cells. Typically, this layer binds the skin to underlying structures, insulates the body from cold, and stores energy in the form of fat. In general the skin forms a protective barrier against the action of physical, chemical, and bacterial agents on the deeper tissues. This means that tissues belonging, e.g. to the oral cavity or the vaginal region or mucous membranes do not belong to the skin. In a preferred embodiment the term “skin” relates to the outermost layer of the body's covering, i.e. the epidermis. In a more preferred embodiment the term “skin” relates to the stratum corneum of the epidermis. In an even more preferred embodiment the term “skin” relates to the outermost 25 to 30 layers of dead cells of the epidermis. More preferably the term “skin” relates to the outermost 10 layers of dead cell of the epidermis.

In a more preferred embodiment the term “skin” relates to the skin of the foot. The term “skin of the foot” relates to the skin zone of the foot, preferably of the skin zone at the sole of the foot. The skin of the foot typically provides a unique habitat, e.g., for microbes. The surface of the skin of the foot usually differs from other regions of the body in several ways. Typically, the stratum corneum, the keratinized horny layer, is thicker at the soles than anywhere else on the body, for example about 0.5 mm. Therefore, the skin of the foot, preferably the skin zone at the sole of the foot, can usually maintain a high moisture content. This means that it is typically less permeable to both ingress and egress of nutrients and fluids than other skin surfaces. Conventionally, the structure of the skin surface of the foot differs in both pattern and distribution from that seen elsewhere on the body. In general, there are no sebaceous glands on the soles or on the dorsum of the foot. Typically, there are no aprocine sweat glands on the foot and no hair follicles on the soles or the terminal phalanges. As is generally known, the skin of the foot is supplied with large numbers of eccrine sweat glands, which typically respond to thermal and mental stimuli differently from the eccrine glands on the rest of body surface. In general, the skin surface of the foot forms a protective barrier against the action of physical, chemical, and bacterial agents on the deeper tissues. Conventially, the pH of the skin zone of the foot is slightly higher than that of other skin areas. Preferably the pH of the skin at the sole of the foot is slightly higher than that of other skin areas. In a preferred embodiment the pH of the skin zone of the foot is higher than 5.0, more preferably higher than 5.5, even more preferably higher than 6.0 and most preferably higher than 6.5.

The term “suppress” in connection with the biosynthesis of isovaleric acid or other odorous compounds means that the formation of isovaleric acid or the other compounds by skin microorganisms, when contacted with a microorganism according to the invention, is stopped or decreased. A “stopped formation” means that isovaleric acid or the other compound is not detectable in a mixture containing a microorganism which is capable of generating isovaleric acid or the other compound and a microorganism according to the invention in the presence of a precursor of isovaleric acid or of the other compound. A “decreased formation” means that the amount of isovaleric acid or the other compounds is reduced in a mixture containing a microorganism which is capable of generating isovaleric acid or the other compound and a microorganism according to the invention in the presence of a precursor of isovaleric acid or of the other compound in comparison to a mixture in which the microorganism according to the invention is not present. The term “reduced” in connection with the biosynthesis of isovaleric acid or the other compound means that the amount of isovaleric acid or the other compound in a mixture containing a microorganism which is capable of generating isovaleric acid or the other compound and a microorganism according to the invention in the presence of a precursor of isovaleric acid or the other compounds is less than 95%, 90%, 80%, 70%, 60%, 50%, 40%, 30%, 20%, 10%, 5%, more preferably less than 3% and most preferably less than 2% of the amount of isovaleric acid or the other compound present in a mixture in which the microorganism according to the invention is not present.

The capability of a microorganism according to the invention to suppress the biosynthesis of isovaleric acid can be determined in an assay as described in the following.

Briefly, such an assay comprises the following steps:
mixing a microorganism which should be tested for its capability to suppress the biosynthesis of isovaleric acid with a microorganism which is able to generate isovaleric acid and with a precursor of isovaleric acid,
incubating the mixture under conditions allowing the generation of isovaleric acid;
extracting short fatty acids from the supernatant of the mixture; and
detecting odor release by the occurrence of isovaleric acid.

The mixing of the components may be carried out in any suitable proportion and in any suitable buffer, known to the person skilled in the art. In a preferred embodiment a microorganism which is able to generate isovaleric acid thereof is cultivated under conditions known by the skilled person to be suitable. Preferably it is aerobically cultivated in BHl broth at 37° C. The cultivation may be carried out, e.g., for 10 to 40 h, preferably for 20 to 35 h and even more preferably for 24 h. The cultivation may be carried out in any vessel known to be suitable to the skilled artisan. Preferably, it is carried out in a shaking glass flask. As volume for the aerobic cultivation any volume suitable can be used, preferably a volume of 1 to 50 ml, more preferably 5 to 40 ml, even more preferably 10 to 30 ml, and most preferably 20 ml is used. As a further step an amount or volume known to be suitable to the skilled artisan may be used as inoculum for a further cultivation. As volume for the aerobic cultivation any volume suitable can be used, preferably a volume of 1 to 50 μl, more preferably 5 to 40 μl, even more preferably 10 to 30 μl, and most preferably 15 μl of the first culture are used as inoculum for a further cultivation. In a most preferred embodiment 15 μl of a 24 h preculture of volume of 20 ml are used as inoculum for a further cultivation. As further step a microorganism which is able to generate isovaleric acid may be cultivated under conditions known by the skilled person to be suitable. Preferably it is aerobically cultivated in BHl broth at 37° C. The cultivation may be carried out, e.g., for 10 to 40 h, preferably for 20 to 35 h and even more preferably for 24 h. As volume for the aerobic cultivation any volume suitable can be used, preferably a volume of 1 to 50 ml, more preferably 5 to 40 ml, even more preferably 10 to 30 ml, and most preferably 20 ml is used. The cultivation may be carried out in any vessel known to be suitable to the skilled artisan. Preferably, the incubation may be carried out under shaking, more preferably, the incubation is carried out under vigorous shaking, e.g. 160 rpm on a reciprocal shaker. The microorganism which is able to generate isovaleric acid is subsequently separated from the culture medium by any suitable method, e.g. the culture of said microorganism can be centrifuged, for example at 4000×g for 5 min. As a further step the obtained microorganism may be washed by any suitable means known to the person skilled in the art, preferably an obtained cell pellet is washed one to several times in a buffer, e.g. a PBS-buffer, pH 8.0. As a further step, the obtained cells may be resuspended in any suitable buffer, known to the person skilled in the art, preferably an obtained cell pellet is resuspended in, e.g. 20 ml of a phosphate (for instance of 60 mM), for example, a PBS-buffer, pH 8.0.

The microorganism which should be tested for its capability to suppress the biosynthesis of isovaleric acid is cultivated under conditions known by the skilled person to be suitable. Preferably, it may cultivated under anaerobic conditions in, e.g., MRS broth at 37° C. More preferably, it may be cultivated in Eppendorf tubes which are closed. The cultivation may be carried out for any time suitable, for instance for 1 to 3 days, preferably for 30 to 60 h, more preferably for 40 to 50 h and even more preferably for 48 h. The cultivation may be started with bacteria in any form known to be suitable to the skilled artisan. Preferably, the cultivation may be started from a −80° C. freezing culture. As volume for the anaerobic cultivation any volume suitable can be used, preferably a volume of 1 to 1000 μl, more preferably of 10 to 500 μl, even more preferably of 100 to 300 μl, and most preferably of 150 μl is used. The microorganism which should be tested for its capability to suppress the biosynthesis of isovaleric acid is subsequently separated from the culture medium by any suitable method, e.g. the culture of said microorganism can be centrifuged, for example at 4000×g for 15 min. As a further step the obtained microorganism may be washed by any suitable means known to the person skilled in the art, preferably an obtained cell pellet is washed one to several times in a buffer, preferably a phosphate puffer, more preferably a 60 mM phosphate buffer, e.g. a PBS-buffer, pH 8.0. As a further step, the obtained cells may be resuspended in any suitable buffer, known to the person skilled in the art, preferably an obtained cell pellet is resuspended in, e.g. 200 μl of a phosphate buffer (for instance of 60 mM), for example, a PBS-buffer, pH 8.0.

For the assay cells of the microorganism which is able to generate isovaleric acid, preferably washed cells, are mixed with isovaleric acid precursors in any suitable proportion known to the person skilled in the art. In a preferred embodiment, 1 to 500 μl of cells are used, more preferably, 5 to 200 μl, even more preferably 10 to 100 μl and most preferably 15 μl are used. The mixing may be carried out in any suitable buffer known to the person skilled in the art, e.g. a phosphate buffer. Preferably the mixing may be carried out in a 60 mM phosphate buffer, pH 8.0. The isovaleric acid precursor may be used in any suitable amount or concentration known to the skilled artisan, e.g. in a concentration of 1 mM to 100 mM, preferably of 2 mM to 50 mM, more preferably of 5 mM to 20 mM and most preferably of 10 mM. As isovaleric acid precursors preferably branched-chain amino acids are used. More preferably leucine or α-ketoglutarate are used. Even more preferably 5 mM L-leucine and 10 mM α-ketoglutarate are used. To such a mixture cells of a culture of a microorganism which should be tested for the capability to suppress the biosynthesis of isovaleric acid may be added in a suitable amount, known to the skilled artisan. Preferably, 1 to 1000 μl are added, more preferably 5 to 500 μl, even more preferably 10 to 250 μl and most preferably 100 μl are added. As a control any suitable buffer or medium, for instance, PBS-buffer or MRS medium in a suitable, corresponding amount may be added to the mixture as characterized herein above. The samples are incubated under conditions allowing the generation of isovaleric acid. Such conditions are known by the skilled person. “Conditions allowing the generation of isovaleric acid” means conditions which are known to the person skilled in the art to allow a microorganism to generate isovaleric acid, as can, for example, be verified in a control in which only a microorganism which is able to generate isovaleric acid is present, but no microorganism capable of suppressing the biosynthesis of isovaleric acid. More preferably, the samples are incubated at 30° C. under aerobic conditions, for example, for 5 to 40 h, even more preferably 7 to 35 h, 10 to 30 h and most preferably for 24 h. Preferably, the incubation may be carried out under shaking, more preferably, the incubation is carried out under vigorous shaking, e.g. 160 rpm on a reciprocal shaker. Afterwards the cells may be centrifuged, e.g. at 4000×g for 5 min, and the supernatant may be acidified, for example with 6 M HCl. Subsequently short chain fatty acids can be extracted with any method known to the person skilled in the art, preferably with 3×150 μl CHCl₃. The extract may further be concentrated, e.g. under nitrogen, to a volume of, e.g., 10 μl.

The presence of isovaleric acid or can be detected by methods known to the person skilled in the art. Preferably, it is determined by GC/MS analysis, e.g. with a Hewlett-Packard GC 5980 series II/MSD 5971 system equipped with a split/splitless injector and a FFAP column. In a preferred embodiment, a small volume, e.g. 1 μl, of an odorous solution or an extract as described herein above may be injected in a GC/MSD equipped, for instance, with a FFAP column, with, e.g. 30 m, 0.5 mm ID, 0.53 μm film thickness, in a splittless mode. Suitable injector and detector temperatures, known to the person skilled in the art, are chosen. Preferably, injector and detector temperatures of 150° C. to 220° C. are chosen. For a sensible separation of short fatty acids suitable temperature conditions known to the person skilled in the art are selected. Preferably, the temperature conditions for a sensible separation of short fatty acids may be 2 min at 150° C. followed by a ramp to the final temperature of 220° C. at 15° C./min. This temperature may be held for 1 to 100 min, preferably for 5 to 500 min and most preferably for 3 min. The column flow may be set according to the conditions known to the person skilled in the art. Preferably, the column flow may be set to 30 cm/s. As a carrier gas any suitable gas known to the person skilled in the art may be used. Preferably, helium may be used. The identification of isovaleric acid may be carried out by comparison of unknown spectra to a pure commercial standards. As an additional identification parameter, for instance, the relative chromatographic retention time can be used. A microorganism is regarded as being able to suppress the biosynthesis of isovaleric acid if the amount of isovaleric acid detected in such an odor release assay with at least one such microorganism is not more than 95%, 90%, 80%, 70%, 60%, 50%, 40%, 30%, 20%, 10%, preferably not more than 5%, more preferably not more than 3% and most preferably not more than 2% of the amount of isovaleric acid that is detectable in a mixture in which the microorganism according to the invention is not present.

The described assay may also be used to identify microorganisms which are capable of suppressing the biosynthesis of isovaleric acid.

In a preferred embodiment the microorgansim which is able to prevent the generation of foot malodor by skin microorganisms, or which is able to suppress the biosynthesis of isovaleric acid, is able to inhibit the growth of malodor generating skin microorganisms. The term “inhibit” in connection with the growth of foot malodor generating skin microorganisms means that the growth of one or more of these microorganisms is decreased when contacted with a microorganism according to the invention.

In a further preferred embodiment, the microorganism of the present invention inhibits the growth of at least one foot malodor generating skin microorganism, preferably of the major representative of the foot malodor generating skin microorganisms, i.e. Micrococcus spec. In a further preferred embodiment, the microorganism of the present invention specifically inhibits the growth of such a microorganism, preferably of Micrococcus spec. “Specifically” preferably means that it inhibits the growth of such a microorganism, preferably of Micrococcus spec, but does not significantly or only to a minor degree inhibit the growth of other microorganisms, in particular of those microorganisms which belong to the resident skin micro flora of the foot. The term “resident skin micro flora of the foot” refers to a flora of aerobic microorganisms which can be found on feet skin, preferably human feet skin. Typically, the skin micro flora of the foot is similar to that seen on other skin sites. Conventionally, bacteria are most abundant, and the predominant bacteria are, for example, Micrococcaceae and, most typcically, Staphylococcus epidermidis (coagulase negative) as well as, e.g., coryneform bacteria, diphteroids (Tachibana D K. Microbiology of the foot. Annu Rev Microbiol. 1976;30:351-375. Marshall J, Leeming J P, Holland K T. The cutaneous microbiology of normal human feet. J Appl Bacteriol. 1987 Feburary; 62(2):139-146). Generally, the range of aerobic bacteria is, e.g., 10² to 10⁶ colony forming units/cm². Typically, on the foot the bacteria exist on the skin surface itself. As is generally know, there is no evidence availabe that bacteria exist below the epidermal layers of normal undamaged skin.

More preferably, the term “specifically” means that the degree of inhibition on a foot malodor generating skin microorganism, preferably Micrococcus, is much higher than the degree of inhibition on another microorganism, in particular a microorganism of the resident skin micro flora of the foot. Particularly preferred, the term “specifically” means that in a suitable growth assay known to the person skilled in the art the proliferation of the foot malodor generating skin microorganism, preferably Micrococcus, in the presence of the microorganism of the present invention is at the most 50% of the proliferation of another microorganism, in particular another microorganism of the resident skin micro flora of the foot in the presence of the microorganism of the present invention. Preferably, the proliferation of the foot malodor generating skin microorganism, preferably Micrococcus spec., is 40%, 30%, 20%, 10%, more preferably 5% and most preferably 0% of the proliferation of another microorganism, in particular another microorganism of the resident skin micro flora of the foot, in the presence of a microorganism of the present invention. The specific inhibition of Micrococcus spec. is indicated in Examples 4 and 5, which show by way of illustration that Micrococcus spec. is inhibited, whereas Staphylococcus epidermidis is not inhibited by a microorganism according to the present invention in an in vitro liquid assay. In a preferred embodiment the microorganism of the present invention inhibits the growth of Micrococcus spec. but does not inhibit the growth of Staphylococcus epidermidis.

A decreased growth means preferably a decrease in proliferation, i.e. cell divisions per time unit. Alternatively, the term “inhibits” also refers to a decrease in size of individual cells. Bacterial cell size can be assessed by flow cytometry (e.g. Becton-Dickinson FACSort flow cytometer, San José, Calif.) after staining with the stain SYBR Green I (Molecular Probes, USA). Bacteria cell size is assessed in Side-Angle Light Scatter (SSC) mode. A decreased growth thus means a decrease in biomass production per time unit.

The decrease of growth of the malodor generating skin microorganisms can preferably be observed in vitro, more preferably in an assay in which a microorganism according to the invention is contacted with one or more malodor generating skin microorganisms and the growth of the(se) malodor generating skin microorganism(s) is determined. The growth can be determined by counting the numbers of cells/colonies after different time intervals of incubation and can be compared with a control which does not contain a microorganism according to the invention, thereby allowing to determine whether there is a decrease in growth.

An in vitro assay for determining the inhibition of growth is described in the Examples and comprises a so-called “in vitro hole plate assay”. In brief, such an assay comprises the following steps:
cultivation of at least one malodor generating skin microorganism and evenly spreading it/them on a prepared agar plate containing a suitable agar medium for growth, and preferably detection, of the respective microorganism(s);
providing holes in the inoculated agar plate;
filling the holes with precultured cells of a microorganism according to the invention;
incubating the agar plates for an appropriate amount of time and under conditions allowing growth of the malodor generating skin microorganisms; and
determining the growth of the microorganism(s) of the malodor generating skin microorganism flora surrounding the holes containing a microorganism according to the invention and comparing it to the growth of the microorganism(s) surrounding a hole which does not contain a microorganism according to the invention.

The determination of the growth in the last step may be effected by available means and methods for determining the number of cells and/or colonies, e.g. by staining with an appropriate dye and/or optical means such as densitometry and counting the cells/colonies under the microscope.

The described assay may also be used to identify microorganisms which are capable of inhibiting the growth of foot malodor generating skin microorganisms.

More preferably, the inhibition of growth of the foot malodor generating skin microorganism can be determined in an “in vitro liquid assay”. Such an assay is described in the Examples and, briefly, comprises the following steps:
cultivation of at least one foot malodor generating skin microorganism in a liquid culture;
applying an aliquot of a liquid culture of the microorganism according to the invention and an aliquot of a liquid culture of the foot malodor generating skin microorganism to a culture medium allowing the growth of the microorganism of the transient pathogenic skin micro flora;
co-cultivation of the microorganism according to the invention and the foot malodor generating skin microorganism in a liquid culture;
transferring an aliquot of the co-cultivation liquid culture to an agar plate, containing an appropriate growth medium;
incubation of the agar plates for a period of time and under conditions allowing the growth of the foot malodor generating skin microorganism;
determining the growth of the foot malodor generating skin microorganism by quantification of the colony forming units and comparing it to the growth of the microorganism(s) in a control in which no microorganism of the invention was applied.

Even more preferably, the growth of the malodor generating skin microorganism flora can also be observed in an “in situ skin assay”. Such an assay comprises the following steps:
cultivation of at least one malodor generating skin microorganism and evenly spreading it/them on an area of skin of a test individual;
applying an aliquot of a microorganism according to the invention in a punctual area within the area on which the malodor generating skin microorganism flora has been spread;
incubating the skin for an amount of time sufficient to allow growth of the malodor generating skin microorganisms;
transferring the upper skin layers, including the microorganisms comprised in these, to an agar plate containing an appropriate growth medium;
incubation of the agar plates for a period of time and under conditions allowing the growth of the malodor generating skin microorganisms;
determining the growth of the malodor generating skin microorganism flora surrounding the area at which the microorganism according to the invention was applied and comparing it to the growth of the microorganism(s) in a control in which no microorganism of the invention was applied.

The area of skin used for this assay may be any suitable area of skin of an individual, preferably of a human individual. In a preferred embodiment it is an area of skin on the foot of a human individual. The size of the area is not decisive, preferably it is about 1 to 40 cm², more preferably 5 to 20 cm², even more preferably 5 to 10 cm², e.g. about 5, 6, 7, 8, 9 or 10 cm.

The malodor generating skin microorganisms are evenly distributed on the area, preferably in a density of approximately 10² cfu/cm²-10³ cfu/cm². The microorganism(s) spread on the skin are air dried and an aliquot of a microorganism according to the invention is applied in a punctual manner within the area. This can be achieved by means known to the person skilled in the art. For example, the microorganisms according to the invention are centrifuged (15 min, 4000×g). The cell pellet is washed two times with K/Na-buffer (each 1 ml). Cells are resuspended in 200 μl K/Na buffer and 10 μl of prepared microorganisms are punctual applied on the pre-inoculated skin area with a micro pipet.

The incubation of the skin preferably takes place at room temperature for, e.g., two hours. The transfer of the upper skin layers, including the microorganisms comprised therein, may, e.g., be effected with the help of an adhesive tape stripe. The agar plates to which the upper skin layers have been transferred are incubated at a temperature allowing growth of the malodor generating skin microorganisms to be tested and contain a growth medium known to support growth of this (these) microorganism(s). The incubation typically takes place for about 24 hours.

The growth of the microorganism(s) can be detected by methods known to the person skilled in the art. Preferably, it is determined by densitometry or by counting the colonies formed in the neighborhood of the point at which an aliquot of the microorganism of the invention was applied. Bacterial cell size can be assessed by flow cytometry (e.g. Becton-Dickinson FACSort flow cytometer, San José, Calif.) after staining with the stain SYBR Green I (Molecular Probes, USA). Bacteria cell size is assessed in Side-Angle Light Scatter (SSC) mode.

A microorganism is regarded to inhibit the growth of one or more foot malodor generating skin microorganisms if it leads to a decrease of growth of at least one such microorganism in an in vitro hole plate assay of at least 5%, preferably of at least 10%, 20%, 30%, 40%, 50%, 60%, 70%, 80%, or 90%, more preferably of at least 95% and even more preferably of at least 99% and most preferably of 100% in comparison to a control to which no microorganism has been added.

More preferably, a microorganism is regarded to inhibit the growth of one or more foot malodor generating skin microorganisms if it leads to a decrease of growth of at least one such microorganism in an in vitro liquid assay of at least 5%, preferably of at least 10%, 20%, 30%, 40%, 50%, 60%, 70%, 80%, or 90%, more preferably of at least 95% and even more preferably of at least 99% and most preferably of 100% in comparison to a control to which no microorganism has been added.

Even more preferably, a microorganism is regarded as inhibiting the growth of one or more foot malodor generating skin microorganisms if it leads to a decrease of growth of at least one such microorganism in an in situ skin assay of at least 5%, preferably of at least 10%, 20%, 30%, 40%, 50%, 60%, 70%, 80%, or 90%, more preferably of at least 95% and even more preferably of at least 99% and most preferably of 100%. The test for determining whether a microorganism inhibits or does not inhibit the growth of a foot malodor generating skin microorganisms, e.g. Micrococcus spec., is preferably an in vitro and/or an in situ test as described herein-above, more preferably a test as described in the Examples.

In a preferred embodiment the odor generating microorganism which is able to generate isovaleric acid to be used in the assays described herein above, belongs to the genus Micrococcus or Propionibacterium. More preferably, the odor generating microorganism is Micrococcus spec., or Propionibacterium freudenreichii. Most preferably, the odor generating microorganism to be used in the assays as described herein above is Propionibacterium freudenreichii subsp. shermanli TL34 (ATCC 9614).

Microorganisms which produce compounds leading to the typical smell of foot maldodor can be isolated as described in the Examples and according to methods known to the person skilled in the art.

In a particularly preferred embodiment the microorganism of the present invention is a microorganism belonging to the group of lactic acid bacteria. The term “microorganism belonging to the group of lactic acid bacteria” encompasses (a) microorganism(s) which belong(s) to bacteria, in particular belonging to gram-positive fermentative eubacteria, more particularly belonging to the family of lactobacteriaceae including lactic acid bacteria. Lactic acid bacteria are from a taxonomical point of view divided up into the subdivisions of Streptococcus, Leuconostoc, Pediococcus, Lactococcus and Lactobacillus. The microorganism of the present invention is preferably a Lactobacillus species or a Lactococcus species or a Leuconostoc species. Members of the lactic acid bacteria group normally lack porphyrins and cytochromes, do not carry out electron-transport phosphorylation and hence obtain energy only by substrate-level phosphorylation. I.e. in lactic acid bacteria ATP is synthesized through fermentation of carbohydrates. All of the lactic acid bacteria grow anaerobically, however, unlike many anaerobes, most lactic acid bacteria are not sensitive to oxygen and can thus grow in its presence as well as in its absence. Accordingly, the bacteria of the present invention are preferably aerotolerant anaerobic lactic acid bacteria, preferably belonging to the genus of Lactobacillus, Lactococcus or Leuconostoc.

The lactic acid bacteria of the present invention are preferably rod-shaped or spherical, varying from long and slender to short bent rods, are moreover preferably immotile and/or asporogenous and produce lactic acid as a major or sole product of fermentative metabolism. The genus Lactobacillus to which the microorganism of the present invention belongs in a preferred embodiment is divided up by the following characteristics into three major subgroups, whereby it is envisaged that the Lactobacillus species of the present invention can belong to each of the three major subgroups:

(a) homofermentative lactobacilli
(i) producing lactic acid, preferably the L-, D- or DL-isomer(s) of lactic acid in an amount of at least 85% from glucose via the Embden-Meyerhof pathway;
(ii) growing at a temperature of 45° C., but not at a temperature of 15° C.;
(iii) being long-rod shaped; and
(iv) having glycerol teichoic acid in the cell wall;

(b) homofermantative lactobacilli
(i) producing lactic acid, preferably the L- or DL-isomer(s) of lactic acid via the Embden-Meyerhof pathway;
(ii) growing at a temperature of 15° C., showing variable growth at a temperature of 45° C.;
(iii) being short-rod shaped or coryneform; and
(iv) having ribitol and/or glycerol teichoic acid in their cell wall;

(c) heterofermentative lactobacilli
(i) producing lactic acid, preferably the DL-isomer of lactic acid in an amount of at least 50% from glucose via the pentose-phosphate pathway;
(ii) producing carbondioxide and ethanol
(iii) showing variable growth at a temperature of 15° C. or 45° C.;
(iv) being long or short rod shaped; and
(v) having glycerol teichoic acid in their cell wall.

Based on the above-described characteristics, the microorganisms of the present invention can be classified to belong to the group of lactic acid bacteria, particularly to the genus of Lactobacillus. By using classical systematics, for example, by reference to the pertinent descriptions in “Bergey's Manual of Systematic Bacteriology” (Williams & Wilkins Co., 1984), a microorganism of the present invention can be determined to belong to the genus of Lactobacillus. Alternatively, the microorganisms of the present invention can be classified to belong to the genus of Lactobacillus by methods known in the art, for example, by their metabolic fingerprint, i.e. a comparable overview of the capability of the microorganism(s) of the present invention to metabolize sugars or by other methods described, for example, in Schleifer et al., System. Appl. Microb., 18 (1995), 461-467 or Ludwiq et al., System. Appl. Microb., 15 (1992), 487-501. The microorganisms of the present invention are capable of metabolizing sugar sources which are typical and known in the art for microorganisms belonging to the genus of Lactobacillus.

The affiliation of the microorganisms of the present invention to the genus of Lactobacillus can also be characterized by using other methods known in the art, for example, using SDS-PAGE gel electrophoresis of total protein of the species to be determined and comparing them to known and already characterized strains of the genus Lactobacillus. The techniques for preparing a total protein profile as described above, as well as the numerical analysis of such profiles, are well known to a person skilled in the art. However, the results are only reliable insofar as each stage of the process is sufficiently standardized. Faced with the requirement of accuracy when determining the attachment of a microorganism to the genus of Lactobacillus, standardized procedures are regularly made available to the public by their authors such as that of Pot et al., as presented during a “workshop” organized by the European Union, at the University of Ghent, in Belgium, on Sep. 12 to 16, 1994 (Fingerprinting techniques for classification and identification of bacteria, SDS-PAGE of whole cell protein). The software used in the technique for analyzing the SDS-PAGE electrophoresis gel is of crucial importance since the degree of correlation between the species depends on the parameters and algorithms used by this software. Without going into the theoretical details, quantitative comparison of bands measured by a densitometer and normalized by a computer is preferably made with the Pearson correlation coefficient. The similarity matrix thus obtained may be organized with the aid of the UPGMA (unweighted pair group method using average linkage) algorithm that not only makes it possible to group together the most similar profiles, but also to construct dendograms (see Kersters, Numerical methods in the classification and identification of bacteria by electrophoresis, in Computer-assisted Bacterial Systematics, 337-368, M. Goodfellow, A. G. O'Donnell Ed., John Wiley and Sons Ltd, 1985).

Alternatively, the affiliation of said microorganisms of the present invention to the genus of Lactobacillus can be characterized with regard to ribosomal RNA in a so called Riboprinter.RTM. More preferably, the affiliation of the newly identified species of the invention to the genus Lactobacillus is demonstrated by comparing the nucleotide sequence of the 16S ribosomal RNA of the bacteria of the invention, or of their genomic DNA which codes for the 16S ribosomal RNA, with those of other genera and species of lactic acid bacteria known to date. Another preferred alternative for determining the attachment of the newly identified species of the invention to the genus Lactobacillus is the use of species-specific PCR primers that target the 16S-23S rRNA spacer region. Another preferred alternative is RAPD-PCR (Nigatu et al. in Antonie van Leeuwenhoek (79), 1-6, 2001) by virtue of that a strain specific DNA pattern is generated which allows to determine the affiliation of an identified microorganisms in accordance with the present invention to the genus of Lactobacillus. Further techniques useful for determining the affiliation of the microorganism of the present invention to the genus of Lactobacillus are restriction fragment length polymorphism (RFLP) (Giraffa et al., Int. J. Food Microbiol. 82 (2003), 163-172), fingerprinting of the repetitive elements (Gevers et al., FEMS Microbiol. Lett. 205 (2001) 31-36) or analysis of the fatty acid methyl ester (FAME) pattern of bacterial cells (Heyrman et al., FEMS Microbiol. Lett. 181 (1991), 55-62). Alternatively, lactobacilli can be determined by lectin typing (Annuk et al., J. Med. Microbiol. 50 (2001), 1069-1074) or by analysis of their cell wall proteins (Gatti et al., Lett. Appl. Microbiol. 25 (1997), 345-348.

In a preferred embodiment of the present application the microorganism is a probiotic microorganism. The term “probiotic” in the context of the present invention means that the microorganism has a beneficial effect on health if it is topically applied to the skin. Preferably, a “probiotic” microorganism is a live microorganism which, when topically applied to the skin, e.g. of the foot, is beneficial for health of this tissue. Most preferably this means that the microorganism has a positive effect on the micro flora of the skin.

In a preferred embodiment the microorganism of the present invention belongs to the species of Lactobacillus plantarum, Lactobacillus curvatus, Lactobacillus delbrückii (preferably Lactobacillus delbrückii delbrückii), Lactobacillus brevis, Lactococcus brevis, Lactococcus lactis or Leuconostoc mesenteroides. However, the lactic acid bacteria of the present invention are not limited thereto.

In a particularly preferred embodiment of the present invention the microorganism of the present invention is selected from the group consisting of Lactobacillus brevis, Lactobacillus plantarum, Lactobacillus curvatus, Lactobacillus delbrückii delbrückii or Leuconostoc mesenteroides being deposited at the DSMZ by the BASF Future Business GmbH, 4. Gartenweg-Z25, 67063 Ludwigshafen, Germany under the accession number DSM 17599 (Lactobacillus brevis, LB-FG-0001), DSM 17600 (Lactobacillus plantarumg, LB-FG-0002), DSM 17601 (Lactobacillus curvatus, LB-FG-0003), DSM 17602 (Leuconostoc mesenteroides, LB-FG-0004), DSM 17603 (Lactobacillus plantarum, LB-FG-0005), DSM 17604 (Lactobacillus delbrückii delbrückii, LB-FG-0006), DSM 17605 (Lactobacillus delbrückii delbrückii, LB-FG-0007), DSM 17606 (Lactobacillus plantarum, LB-FG-0008) and DSM 17607 (Lactobacillus brevis, LB-FG-0009).

The invention also relates to a mutant or derivative of the above-mentioned deposited lactic acid bacteria strains wherein said mutants or derivatives have retained the capability to prevent the generation of foot malodor by skin microorganisms, more preferably the capability to suppress the biosynthesis of isovaleric acid and/or to inhibit the growth of foot malodor generating skin microorganisms.

The term “Lactobacillus brevis, Lactobacillus plantarum, Lactobacillus curvatus, Lactobacillus delbrückii delbrückii or Leuconostoc mesenteroides being deposited at the DSMZ” relates to cells of a microorganism belonging to the species Lactobacillus brevis, Lactobacillus plantarum, Lactobacillus curvatus, Lactobacillus delbrückii delbrückii or Leuconostoc mesenteroides deposited at the Deutsche Sammlung für Mikroorganismen und Zellkulturen (DSMZ) on Sep. 22, 2005 by the BASF Future Business GmbH, 4. Gartenweg-Z25, 67063 Ludwigshafen, Germany and having the following deposit numbers: DSM 17599 (Lactobacillus brevis, LB-FG-0001), DSM 17600 (Lactobacillus plantarum, LB-FG-0002), DSM 17601 (Lactobacillus curvatus, LB-FG-0003), DSM 17602 (Leuconostoc mesenteroides, LB-FG-0004), DSM 17603 (Lactobacillus plantarum, LB-FG-0005), DSM 17604 (Lactobacillus delbrückii delbrückii, LB-FG-0006), DSM 17605 (Lactobacillus delbrückii delbrückii, LB-FG-0007), DSM 17606 (Lactobacillus plantarum, LB-FG-0008) and DSM 17607 (Lactobacillus brevis, LB-FG-0009). The DSMZ is located at the Mascheroder Weg 1b, D-38124 Braunschweig, Germany. The aforementioned deposits were made pursuant to the terms of the Budapest treaty on the international recognition of the deposit of microorganisms for the purposes of patent procedures. In a particular preferred embodiment the microorganisms of the present invention are “isolated” or “purified”. The term “isolated” means that the material is removed from its original environment, e.g. the natural environment if it is naturally occurring, or the culture medium if it is cultured. For example, a naturally-occurring microorganism, preferably a lactic acid bacterium, separated from some or all of the coexisting materials in the natural system, is isolated. Such a microorganism could be part of a composition, and is to be regarded as still being isolated in that the composition is not part of its natural environment.

The term “purified” does not require absolute purity; rather, it is intended as a relative definition. Individual microorganisms obtained from a library have been conventionally purified to microbiological homogeneity, i.e. they grow as single colonies when streaked out on agar plates by methods known in the art. Preferably, the agar plates that are used for this purpose are selective for a lactic acid bacterium, preferably Lactobacillus species. Such selective agar plates are known in the art.

In another aspect the present invention relates to an inactivated form of the microorganism of the present invention, which is, e.g., thermally inactivated or lyophilized, but which retains the property of retained their capability to suppress the biosynthesis of isovaleric acid.

According to the present invention the term “inactivated form of the microorganism of the present invention” includes a dead or inactivated cell of the microorganism of the present invention, preferably of a lactic acid bacterium disclosed herein, which is no longer capable to form a single colony on a plate specific for microorganisms belonging lactic acid bacteria. Said dead or inactivated cell may have either an intact or broken cell membrane. Methods for killing or inactivating cells of the microorganism of the present invention are known in the art. EI-Nezami et al., J. Food Prot. 61 (1998), 466-468 describes a method for inactivating Lactobacillus species by UV-irradiation. Preferably, the cells of the microorganism of the present invention are thermally inactivated or lyophilised. Lyophilisation of the cells of the present invention has the advantage that they can be easily stored and handled while retaining their property to prevent the generation of foot malodor by skin microorganisms.

Moreover, lyophilised cells can be grown again when applied under conditions known in the art to appropriate liquid or solid media. Lyophilization is done by methods known in the art. Preferably, it is carried out for at least 2 hours at room temperature, i.e. any temperature between 16° C. and 25° C. Moreover, the lyophilized cells of the microorganism of the present invention are stable for at least 4 weeks at a temperature of 4° C. so as to still retain their properties as described above. Thermal inactivation can be achieved by incubating the cells of the microorganism of the present invention for at least 2 hours at a temperature of 170° C. Yet, thermal inactivation is preferably achieved by autoclaving said cells at a temperature of 121° C. for at least 20 minutes in the presence of saturated steam at an atmospheric pressure of 2 bar. In the alternative, thermal inactivation of the cells of the microorganism of the present invention is achieved by freezing said cells for at least 4 weeks, 3 weeks, 2 weeks, 1 week, 12 hours, 6 hours, 2 hours or 1 hour at −20° C. It is preferred that at least 70%, 75% or 80%, more preferably at least 85%, 90% or 95% and particularly preferred at least 97%, 98%, 99% and more particularly preferred, at least 99.1%, 99.2%, 99.3%, 99.4%, 99.5%, 99.6%, 99.7%, 99.8% or at least 99.9% and most particularly preferred 100% of the cells of the inactivated form of the microorganism of the present invention are dead or inactivated, however, they have still the capability to prevent the generation of foot malodor by skin microorganisms. Whether the inactivated form of the microorganism of the present invention is indeed dead or inactivated can be tested by methods known in the art, for example, by a test for viability.

The term “inactivated form of the microorganism of the present invention” also encompasses lysates or fractions of the microorganism of the present invention, preferably of the lactic acid bacteria disclosed herein, wherein said lysates or fractions preferably prevent the generation of foot malodor by skin microorganisms. This suppression can be tested as described herein and in particular as described in the appended Examples. In case, a lysate or fraction of the microorganism of the present invention may prevent the generation of foot malodor by skin microorganisms then the skilled person can, for example, further purify said lysate or fraction by methods known in the art, which are exemplified herein below, so as to remove substances which may prevent the generation of foot malodor by skin microorganisms. This means that, if a lysate or fraction of the microorganism of the present invention may not prevent the generation of foot malodor by skin microorganisms, the skilled person can, for example, further purify said lysate or fraction by methods known in the art, so as to remove substances which may impede the prevention of the generation of foot malodor by skin microorganisms. Afterwards the person skilled in the art can again test said lysate or fraction whether it prevents the generation of foot malodor by skin microorganisms.

According to the present invention the term “lysate” means a solution or suspension in an aqueous medium of cells of the microorganism of the present invention that are broken. However, the term should not be construed in any limiting way. The cell lysate comprises, e.g., macromolecules, like DNA, RNA, proteins, peptides, carbohydrates, lipids and the like and/or micromolecules, like amino acids, sugars, lipid acids and the like, or fractions of it. Additionally, said lysate comprises cell debris which may be of smooth or granular structure. Methods for preparing cell lysates of microorganism are known in the art, for example, by employing French press, cells mill using glass or iron beads or enzymatic cell lysis and the like. In addition, lysing cells relates to various methods known in the art for opening/destroying cells. The method for lysing a cell is not important and any method that can achieve lysis of the cells of the microorganism of the present invention may be employed. An appropriate one can be chosen by the person skilled in the art, e.g. opening/destruction of cells can be done enzymatically, chemically or physically. Non-limiting examples for enzymes and enzyme cocktails are proteases, like proteinase K, lipases or glycosidases; non-limiting examples for chemicals are ionophores, detergents, like sodium dodecyl sulfate, acids or bases; and non-limiting examples of physical means are high pressure, like French-pressing, osmolarity, temperature, like heat or cold. Additionally, a method employing an appropriate combination of an enzyme other than the proteolytic enzyme, an acid, a base and the like may also be utilized. For example, the cells of the microorganism of the present invention are lysed by freezing and thawing, more preferably freezing at temperatures below −70° C. and thawing at temperatures of more than 30° C., particularly freezing is preferred at temperatures below −75° C. and thawing is preferred at temperatures of more than 35° C. and most preferred are temperatures for freezing below −80° C. and temperatures for thawing of more than 37° C. It is also preferred that said freezing/thawing is repeated for at least 1 time, more preferably for at least 2 times, even more preferred for at least 3 times, particularly preferred for at least 4 times and most preferred for at least 5 times.

Accordingly, those skilled in the art can prepare the desired lysates by referring to the above general explanations, and appropriately modifying or altering those methods, if necessary. Preferably, the aqueous medium used for the lysates as described is water, physiological saline, or a buffer solution. An advantage of a bacterial cell lysate is that it can be easily produced and stored cost efficiently since less technical facilities are needed.

Preferably, the term “extract” means a subcellular component of the microorganism of the present invention, e.g., macromolecules, like DNA, RNA, proteins, peptides, carbohydrates, lipids and the like and/or micromolecules, like amino acids, sugars, lipid acids and the like or any other organic compound or molecule, or fractions of it, wherein said extract prevents the generation of foot malodor by skin microorganisms. More preferably, the term “extract” refers to any of the above described subcellular components in a cell-free medium.

In a further preferred embodiment an extract may be obtained by lysing cells according to various methods known in the art for opening/destroying cells, as described herein above and/or as supernatant of a centrifugation procedure of a culture of the microorganism of the present invention in any appropriate liquid, medium or buffer known to the person skilled in the art or of a lysate of such a culture or any other suitable cell suspension. More preferably, the extract may be a purified lysate or cell culture supernatant or any fraction or subportion thereof, wherein said purified lysate or cell culture supernatant or any fraction or subportion thereof prevents the generation of foot malodor by skin microorganisms. Suitable methods for purification of an extract are known to the person skilled in the art and comprise, for example, affinity chromatography, ion-exchange chromatography, size-exclusion chromatography, reversed phase-chromatography, and chromatography with other chromatographic material in column or batch methods, other fractionation methods, e.g., filtration methods, e.g., ultrafiltration, dialysis, dialysis and concentration with size-exclusion in centrifugation, centrifugation in density-gradients or step matrices, precipitation, e.g., affinity precipitations, salting-in or salting-out (ammoniumsulfate-precipitation), alcoholic precipitations or other proteinchemical, molecular biological, biochemical, immunological, chemical or physical.

According to the invention, lysates are also preparations of fractions of molecules from the above-mentioned lysates. These fractions can be obtained by methods known to those skilled in the art, e.g., chromatography, including, e.g., affinity chromatography, ion-exchange chromatography, size-exclusion chromatography, reversed phase-chromatography, and chromatography with other chromatographic material in column or batch methods, other fractionation methods, e.g., filtration methods, e.g., ultrafiltration, dialysis, dialysis and concentration with size-exclusion in centrifugation, centrifugation in density-gradients or step matrices, precipitation, e.g., affinity precipitations, salting-in or salting-out (ammoniumsulfate-precipitation), alcoholic precipitations or other proteinchemical, molecular biological, biochemical, immunological, chemical or physical methods to separate above components of the lysates. In a preferred embodiment those fractions which are more immunogenic than others are preferred. Those skilled in the art are able to choose a suitable method and determine its immunogenic potential by referring to the above general explanations and specific explanations in the examples herein, and appropriately modifying or altering those methods, if necessary.

Accordingly, the term “an inactive form of the microorganism of the present invention” also encompasses filtrates of the microorganism of the present invention, preferably of the lactic acid bacteria disclosed herein, wherein said filtrates preferably prevent the generation of foot malodor by skin microorganisms This suppression can be tested as described herein and in particular as described in the appended Examples. In case, a filtrate of the microorganism of the present invention may not prevent the generation of foot malodor by skin microorganisms then the skilled person can, for example, further purify said filtrate by methods known in the art, which are exemplified herein below, so as to remove substances which may impede the prevention of the generation of foot malodor by skin microorganisms. Afterwards the person skilled in the art can again test said filtrate whether it prevents the generation of foot malodor by skin microorganisms.

The term “filtrate” means a cell-free solution or suspension of the microorganism of the present invention which has been obtained as supernatant of a centrifugation procedure of a culture of the microorganism of the present invention in any appropriate liquid, medium or buffer known to the person skilled in the art. However, the term should not be construed in any limiting way. The filtrate comprises, e.g., macromolecules, like DNA, RNA, proteins, peptides, carbohydrates, lipids and the like and/or micromolecules, like amino acids, sugars, lipid acids and the like, or fractions of it. Methods for preparing filtrates of microorganism are known in the art. In addition, “filtrate” relates to various methods known in the art. The exact method is not important and any method that can achieve filtration of the cells of the microorganism of the present invention may be employed.

The term “an inactive form of the microorganism of the present invention” encompasses any part of the cells of the microorganism of the present invention. Preferably, said inactive form is a membrane fraction obtained by a membrane-preparation. Membrane preparations of microorganisms belonging to lactic acid bacteria can be obtained by methods known in the art, for example, by employing the method described in Rollan et al., Int. J. Food Microbiol. 70 (2001), 303-307, Matsuguchi et al., Clin. Diagn. Lab. Immunol. 10 (2003), 259-266 or Stentz et al., Appl. Environ. Microbiol. 66 (2000), 4272-4278 or Varmanen et al., J. Bacteriology 182 (2000), 146-154. Alternatively, a whole cell preparation is also envisaged.

In another aspect the present invention relates to a composition comprising a microorganism according to the present invention or a mutant, derivative or inactive form of this microorganism as described above. In a preferred embodiment, said composition comprises a microorganism as described above in an amount between 10² to 10¹² cells, preferably 10³ to 10⁸ cells per mg in a solid form of the composition. In case of a liquid form of compositions, the amount of the microorganisms is between 10² to 10¹³ cells per ml. In a further preferred embodiment said compositions are in the form of emulsions, e.g. oil in water or water in oil emulsions, in the form of ointments or in the form of micro-capsules. In case of emulsions, ointments or microcapsules the compositions comprise a microorganism as described herein in an amount between 10² to 10¹³ cells per ml. However, for specific compositions the amount of the microorganism may be different as is described herein.

The term “composition” also includes textile compositions as described further below.

In a still further aspect, the present invention provides a method for the production of a composition for preventing the generation of foot malodor by skin microorganisms comprising the steps of formulating a microorganism according to the invention or a mutant, derivative or inactive form of this microorganism as described above with a cosmetically or pharmaceutical acceptable carrier or excipient.

The term “composition”, as used in accordance with the present invention, relates to (a) composition(s) which comprise(s) at least one microorganism of the present invention or mutant, derivative or inactive form of said microorganism as described above. It is envisaged that the compositions of the present invention which are described herein below comprise the aforementioned ingredients in any combination. It may, optionally, comprise at least one further ingredient suitable for prevening the generation of foot malodor by skin microorganisms. Accordingly, it may optionally comprise any combination of the hereinafter described further ingredients. The term “ingredient suitable for preventing the generation of foot malodor by skin microorganisms” encompasses compounds or compositions and/or combinations thereof which lead to an altered pH value, for example an increased or reduced pH value. In a preferred embodiment said term encompasses compounds or compositions and/or combinations thereof which lead to an increased pH value.

The composition may be in solid, liquid or gaseous form and may be, inter alia, in the form of (a) powder(s), (a) solution(s) (an) aerosol(s), suspensions, emulsions, liquids, elixirs, extracts, tincture or fluid extracts or in a form which is particularly suitable for topical administration. Forms suitable for topical application include, e.g., a deodorant, a paste, an ointment, a pumpspray, a lotion, a gel, a cream, a cream or fluid gel distributed as an aerosol spray, e.g. in a pump-dispenser bottle or as a roll-on, in the form of thick creams distributed in tubes or a grille, in the form of wands, or as a transdermal patch.

Preferably, the composition of the present invention is a cosmetic composition further comprising a cosmetically acceptable carrier or excipient.

The cosmetic composition of the present invention comprises the microorganism of the present invention, mutant, derivative or inactive form thereof as described above in connection with the composition of the invention and further a cosmetically acceptable carrier. Preferably the cosmetic composition of the present invention is for use in topical applications.

The term “cosmetically acceptable carrier” as used herein means a suitable vehicle, which can be used to apply the present compositions to the skin in a safe and effective manner. Such vehicle may include materials such as emulsions, e.g. oil in water or water in oil emulsions, ointments or micro-capsules. The term “safe and effective manner” as used herein, means a sufficient amount to suppress the biosynthesis of isovaleric acid.

In another aspect the present invention relates to a pharmaceutical composition comprising the microorganism of the present invention or a derivative or mutant or an inactive form thereof as described above further comprising a pharmaceutical acceptable carrier or excipient. The pharmaceutical composition preferably is in a form which is suitable for topical administration.

In addition, the present invention relates to the use of a microorganism of the present invention or of a derivative or mutant or an inactive form thereof as described above for the preparation of a composition, preferably a pharmaceutical or cosmetic composition for suppressing foot malodor by preventing the generation of foot malodor by skin microorganisms.

Pharmaceutical compositions comprise a therapeutically effective amount of a microorganism of the present invention or of a derivative or mutant of the present invention or an inactive form of said microorganism of the present invention as described above and can be formulated in various forms, e.g. in solid, liquid, powder, aqueous, lyophilized form.

The pharmaceutical composition may be administered with a pharmaceutically acceptable carrier to a patient, as described herein. In a specific embodiment, the term “pharmaceutically acceptable” means approved by a regulatory agency or other generally recognized pharmacopoeia for use in animals, and more particularly in humans.

The term “carrier” refers to a diluent, adjuvant, excipient, or vehicle with which the therapeutic is administered. Such a carrier is pharmaceutically acceptable, i.e. is non-toxic to a recipient at the dosage and concentration employed. It is preferably isotonic, hypotonic or weakly hypertonic and has a relatively low ionic strength, such as provided by a sucrose solution. Such pharmaceutical carriers can be sterile liquids, such as water and oils, including those of petroleum, animal, vegetable or synthetic origin, such as peanut oil, soybean oil, mineral oil, sesame oil and the like. Saline solutions and aqueous dextrose and glycerol solutions can also be employed as liquid carriers. Suitable pharmaceutical excipients include starch, glucose, sucrose, gelatin, malt, rice, flour, chalk, silica gel, sodium stearate, glycerol monostearate, talc, sodium ion, dried skim milk, glycerol, propylene, glycol, water, ethanol and the like. The composition, if desired, can also contain minor amounts of wetting or emulsifying agents, or pH buffering agents. These compositions can take the form of, e.g., solutions, suspensions, emulsion, powders, sustained-release formulations and the like. Examples of suitable pharmaceutical carriers are described in “Remington's Pharmaceutical Sciences” by E. W. Martin. Some other examples of substances which can serve as pharmaceutical carriers are sugars, such as glucose and sucrose; starches such as corn starch and potato starch; cellulose and its derivatives such as sodium carboxymethycellulose, ethylcellulose and cellulose acetates; powdered tragancanth; malt; gelatin; talc; stearic acids; magnesium stearate; calcium sulfate; calcium carbonate; vegetable oils, such as peanut oils, cotton seed oil, sesame oil, olive oil, corn oil and oil of theobroma; polyols such as propylene glycol, glycerine, sorbitol, manitol, and polyethylene glycol; agar; alginic acids; pyrogen-free water; isotonic saline; cranberry extracts and phosphate buffer solution; skim milk powder; as well as other non-toxic compatible substances used in pharmaceutical formulations such as Vitamin C, estrogen and echinacea, for example. Wetting agents and lubricants such as sodium lauryl sulfate, as well as coloring agents, flavoring agents, lubricants, excipients, tabletting agents, stabilizers, anti-oxidants and preservatives, can also be present. It is also advantageous to administer the active ingredients in encapsulated form, e.g. as cellulose encapsulation, in gelatine, with polyamides, niosomes, wax matrices, with cyclodextrins or liposomally encapsulated.

Generally, the ingredients are supplied either separately or mixed together in unit dosage form, for example, as a dry lyophilised powder or water free concentrate in a hermetically sealed container such as an ampoule or sachette indicating the quantity of active agent.

The pharmaceutical composition of the invention can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

An in vitro assay, e.g. one of those described in the Examples, may optionally be employed to help identify optimal dosage ranges. The precise dose to be employed in the formulation will also depend on the route of administration, and the seriousness of the disease or disorder, and should be decided according to the judgment of the practitioner and each patient's circumstances. The topical route of administration is preferred. Effective doses may be extrapolated from dose-response curves derived from in vitro or (animal) model test systems. Preferably, the pharmaceutical composition is administered directly or in combination with an adjuvant. Adjuvants may be selected from the group consisting of a chloroquine, protic polar compounds, such as propylene glycol, polyethylene glycol, glycerol, EtOH, 1-methyl L-2-pyrrolidone or their derivatives, or aprotic polar compounds such as dimethylsulfoxide (DMSO), diethylsulfoxide, di-n-propylsulfoxide, dimethylsulfone, sulfolane, dimethylformamide, dimethylacetamide, tetramethylurea, acetonitrile or their derivatives. These compounds are added in conditions respecting pH limitations. The composition of the present invention can be administered to a vertebrate. “Vertebrate” as used herein is intended to have the same meaning as commonly understood by one of ordinary skill in the art. Particularly, “vertebrate” encompasses mammals, and more particularly humans.

The term “administered” means administration of a therapeutically effective dose of the aforementioned composition. By “therapeutically effective amount” is meant a dose that produces the effects for which it is administered, preferably this effect is the prevention of the generation of foot malodor by skin microorganisms. The exact dose will depend on the purpose of the treatment, and will be ascertainable by one skilled in the art using known techniques. As is known in the art and described above, adjustments for systemic versus localized delivery, age, body weight, general health, sex, diet, time of administration, drug interaction and the severity of the condition may be necessary, and will be ascertainable with routine experimentation by those skilled in the art.

The methods are applicable to human therapy. The compounds described herein having the desired therapeutic activity may be administered in a physiologically acceptable carrier to a patient, as described herein. Depending upon the manner of administration, the compounds may be formulated in a variety of ways as discussed below. The concentration of the therapeutically active compound in the formulation may vary from about 0.01-100 wt %. The agent may be administered alone or in combination with other treatments.

The administration of the pharmaceutical composition can be done in a variety of ways. The preferable route of administering is the topical route.

The attending physician and clinical factors will determine the dosage regimen. As is well known in the medical arts, dosages for any one patient depends upon many factors, including the patient's size, body surface area, age, the particular compound to be administered, sex, time and route of administration, general health, and other drugs being administered concurrently. A typical dose can be, for example, in the range of 0.001 to 1000 μg; however, doses below or above this exemplary range are envisioned, especially considering the aforementioned factors.

The dosages are preferably given once a week, more preferably 2 times, 3 times, 4 times, 5 times or 6 times a week and most preferably daily and even more preferably, 2 times a day or more often. However, during progression of the treatment the dosages can be given in much longer time intervals and in need can be given in much shorter time intervals, e.g., several times a day. In a preferred case the immune response is monitored using herein described methods and further methods known to those skilled in the art and dosages are optimized, e.g., in time, amount and/or composition. Progress can be monitored by periodic assessment. It is also envisaged that the pharmaceutical compositions are employed in co-therapy approaches, i.e. in co-administration with other medicaments or drugs, for example other drugs for preventing the generation of foot malodor by skin microorganisms.

Topical administration of the cosmetic or pharmaceutical composition of the present invention is useful when the desired treatment involves areas or organs readily accessible by topical administration. For application topically to the skin, the pharmaceutical composition is preferably formulated in the form of a deodorant or a spray (pumpspray or aerosol) or with a paste, an ointment, a lotion, a cream, a gel or a transdermal patch. The cosmetic or pharmaceutical preparations can, depending on the field of use, also be in the form of a foam, gel spray, mousse, suspensions or powders.

The cosmetic or pharmaceutical composition may also be formulated in the form of spray (pumpspray or aerosol). Suitable propellants for aerosols according to the invention are the customary propellants, for example propane, butane, pentane and others.

Alternatively the cosmetic or pharmaceutical composition may also be formulated with a suitable paste comprising the active ingredient suspended in a carrier. Such carriers include, but are not limited to, petroleum, soft white paraffin, yellow petroleum jelly and glycerol.

The cosmetic or pharmaceutical composition may also be formulated with a suitable ointment comprising the active components suspended or dissolved in a carrier. Such carriers include, but are not limited to, one or more of glycerol, mineral oil, liquid oil, liquid petroleum, white petroleum, yellow petroleum jelly, propylene glycol, alcohols, triglycerides, fatty acid esters such as cetyl ester, polyoxyethylene polyoxypropylene compound, waxes such as white wax and yellow beeswax, fatty acid alcohols such as cetyl alcohol, stearyl alcohol and cetylstearylalcohol, fatty acids such as stearic acid, cetyl stearate, lanolin, magnesium hydroxide, kaolin and water. Alternatively, the cosmetic or pharmaceutical composition may also be formulated with a suitable lotion or cream comprising the active components suspended or dissolved in a carrier. Such carriers include, but are not limited to, one or more of mineral oil such as paraffin, vegetable oils such as castor oil, castor seed oil and hydrogenated castor oil, sorbitan monostearat, polysorbat, fatty acid esters such as cetyl ester, wax, fatty acid alcohols such as cetyl alcohol, stearyl alcohol, 2-octyldodecanol, benzyl alcohol, alcohols, triglycerides and water.

Alternatively, the cosmetic or pharmaceutical composition may also be formulated with a suitable gel comprising the active components suspended or dissolved in a carrier. Such carriers include, but are not limited to, one or more of water, glycerol, propyleneglycole, liquid paraffin, polyethylene, fatty oils, cellulose derivatives, bentonite and colloidal silicon dioxide.

The preparations according to the invention may generally comprise further auxiliaries as are customarily used in such preparations, e.g. preservatives, perfumes, antifoams, dyes, pigments, thickeners, surface-active substances, emulsifiers, emollients, finishing agents, fats, oils, waxes or other customary constituents, of a cosmetic or dermatological formulation, such as alcohols, polyols, polymers, foam stabilizers, solubility promoters, electrolytes, organic acids, organic solvents, or silicone derivatives.

The cosmetic or pharmaceutical composition according to the invention may comprise emollients. Emollients may be used in amounts which are effective to prevent or relieve dryness. Useful emollients include, without limitation: hydrocarbon oils and waxes; silicone oils; triglyceride esters; acetoglyceride esters; ethoxylated glyceride; alkyl esters; alkenyl esters; fatty acids; fatty alcohols; fatty alcohol ethers; etheresters; lanolin and derivatives; polyhydric alcohols (polyols) and polyether derivatives; polyhydric alcohol (polyol) esters; wax esters; beeswax derivatives; vegetable waxes; phospholipids; sterols; and amides.

Thus, for example, typical emollients include mineral oil, especially mineral oils having a viscosity in the range of 50 to 500 SUS, lanolin oil, mink oil, coconut oil, cocoa butter, olive oil, almond oil, macadamia nut oil, aloa extract, jojoba oil, safflower oil, corn oil, liquid lanolin, cottonseed oil, peanut oil, purcellin oil, perhydrosqualene (squalene), caster oil, polybutene, odorless mineral spirits, sweet almond oil, avocado oil, calophyllum oil, ricin oil, vitamin E acetate, olive oil, mineral spirits, cetearyl alcohol (mixture of fatty alcohols consisting predominantly of cetyl and stearyl alcohols), linolenic alcohol, oleyl alcohol, octyl dodecanol, the oil of cereal germs such as the oil of wheat germ cetearyl octanoate (ester of cetearyl alcohol and 2-ethylhexanoic acid), cetyl palmitate, diisopropyl adipate, isopropyl palmitate, octyl palmitate, isopropyl myristate, butyl myristate, glyceryl stearate, hexadecyl stearate, isocetyl stearate, octyl stearate, octylhydroxy stearate, propylene glycol stearate, butyl stearate, decyl oleate, glyceryl oleate, acetyl glycerides, the octanoates and benzoates of (C12-C15) alcohols, the octanoates and decanoates of alcohols and polyalcohols such as those of glycol and glycerol, and ricin-oleates of alcohols and poly alcohols such as those of isopropyl adipate, hexyl laurate, octyl dodecanoate, dimethicone copolyol, dimethiconol, lanolin, lanolin alcohol, lanolin wax, hydrogenated lanolin, hydroxylated lanolin, acetylated lanolin, petrolatum, isopropyl lanolate, cetyl myristate, glyceryl myristate, myristyl myristate, myristyl lactate, cetyl alcohol, isostearyl alcohol stearyl alcohol, and isocetyl lanolate, and the like.

Moreover, the cosmetic or pharmaceutical composition according to the invention may also comprise emulsifiers. Emulsifiers (i.e., emulsifying agents) are preferably used in amounts effective to provide uniform blending of ingredients of the composition. Useful emulsifiers include (i) anionics such as fatty acid soaps, e.g., potassium stearate, sodium stearate, ammonium stearate, and triethanolamine stearate; polyol fatty acid monoesters containing fatty acid soaps, e.g., glycerol monostearate containing either potassium or sodium salt; sulfuric esters (sodium salts), e.g., sodium lauryl 5 sulfate, and sodium cetyl sulfate; and polyol fatty acid monoesters containing sulfuric esters, e.g., glyceryl monostearate containing sodium lauryl surfate; (ii) cationics chloride such as N(stearoyl colamino formylmethyl) pyridium; N-soya-N-ethyl morpholinium ethosulfate; alkyl dimethyl benzyl ammonium chloride; diisobutylphenoxytheoxyethyl dimethyl benzyl ammonium chloride; and cetyl pyridium chloride; and (iii) nonionics such as polyoxyethylene fatty alcohol ethers, e.g., monostearate; polyoxyethylene lauryl alcohol; polyoxypropylene fatty alcohol ethers, e.g., propoxylated oleyl alcohol; polyoxyethylene fatty acid esters, e.g., polyoxyethylene stearate; polyoxyethylene sorbitan fatty acid esters, e.g., polyoxyethylene sorbitan monostearate; sorbitan fatty acid esters, e.g., sorbitan; polyoxyethylene glycol fatty acid esters, e.g., polyoxyethylene glycol monostearate; and polyol fatty acid esters, e.g., glyceryl monostearate and propylene glycol monostearate; and ethoxylated lanolin derivatives, e.g., ethoxylated lanolins, ethoxylated lanolin alcohols and ethoxylated cholesterol. The selection of emulsifiers is exemplarly described in Schrader, Grundlagen und Rezepturen der Kosmetika, Hüthig Buch Verlag, Heidelberg, 2^{nd} edition, 1989, 3^{rd} part.

The cosmetic or pharmaceutical composition according to the invention may also include a surfactant. Suitable surfactants may include, for example, those surfactants generally grouped as cleansing agents, emulsifying agents, foam boosters, hydrotropes, solubilizing agents, suspending agents and nonsurfactants (facilitates the dispersion of solids in liquids).

The surfactants are usually classified as amphoteric, anionic, cationic and nonionic surfactants. Amphoteric surfactants include acylamino acids and derivatives and N-alkylamino acids. Anionic surfactants include: acylamino acids and salts, such as, acylglutamates, acylpeptides, acylsarcosinates, and acyltaurates; carboxylic acids and salts, such as, alkanoic acids, ester carboxylic acids, and ether carboxylic acids; sulfonic acids and salts, such as, acyl isethionates, alkylaryl sulfonates, alkyl sulfonates, and sulfosuccinates; sulfuric acid esters, such as, alkyl ether sulfates and alkyl sulfates. Cationic surfactants include: alkylamines, alkyl imidazolines, ethoxylated amines, and quaternaries (such as, alkylbenzyldimethylammonium salts, alkyl betaines, heterocyclic ammonium salts, and tetra alkylammonium salts). And nonionic surfactants include: alcohols, such as primary alcohols containing 8 to 18 carbon atoms; alkanolamides such as alkanolamine derived amides and ethoxylated amides; amine oxides; esters such as ethoxylated carboxylic acids, ethoxylated glycerides, glycol esters and derivatives, monoglycerides, polyglyceryl esters, polyhydric alcohol esters and ethers, sorbitan/sorbitol esters, and triesters of phosphoric acid; and ethers such as ethoxylated alcohols, ethoxylated lanolin, ethoxylated polysiloxanes, and propoxylated polyoxyethylene ethers.

Furthermore, a cosmetic or pharmaceutical composition according to the invention may also comprise a film former. Suitable film formers which are used in accord with the invention keep the composition smooth and even and include, without limitation: acrylamide/sodium acrylate copolymer; ammonium acrylates copolymer; Balsam Peru; cellulose gum; ethylene/maleic anhydride copolymer; hydroxyethylcellulose; hydroxypropylcellulose; polyacrylamide; polyethylene; polyvinyl alcohol; pvm/MA copolymer (polyvinyl methylether/maleic anhydride); PVP (polyvinylpyrrolidone); maleic anhydride copolymer such as PA-18 available from Gulf Science and Technology; PVP/hexadecene copolymer such as Ganex V-216 available from GAF Corporation; acryliclacrylate copolymer; and the like.

Generally, film formers can be used in amounts of about 0.1% to about 10% by weight of the total composition with about 1% to about 8% being preferred and about 0.1 DEG/O to about 5% being most preferred. Humectants can also be used in effective amounts, including: fructose; glucose; glulamic acid; glycerin; honey; maltitol; methyl gluceth-10; methyl gluceth-20; propylene glycol; sodium lactate; sucrose; and the like.

Of course, the cosmetic or pharmaceutical composition of the present invention can also comprise a preservative. Preservatives according to certain compositions of the invention include, without limitation: butylparaben; ethylparaben; imidazolidinyl urea; methylparaben; O-phenylphenol; propylparaben; quaternium-14; quaternium-15; sodium dehydroacetate; zinc pyrithione; and the like.

The preservatives are used in amounts effective to prevent or retard microbial growth. Generally, the preservatives are used in amounts of about 0.1% to about 1% by weight of the total composition with about 0.1% to about 0.8% being preferred and about 0.1% to about 0.5% being most preferred.

A cosmetic or pharmaceutical composition according to the invention may also comprise a perfume. Perfumes (fragrance components) and colorants (coloring agents) well known to those skilled in the art may be used in effective amounts to impart the desired fragrance and color to the compositions of the invention.

Furthermore, a cosmetic or pharmaceutical composition of the present invention may also comprise a wax. Suitable waxes which are useful in accord with the invention include: animal waxes, such as beeswax, spermaceti, or wool wax (lanolin); plant waxes, such as carnauba or candelilla; mineral waxes, such as montan wax or ozokerite; and petroleum waxes, such as paraffin wax and microcrystalline wax (a high molecular weight petroleum wax). Animal, plant, and some mineral waxes are primarily esters of a high molecular weight fatty alcohol with a high molecular weight fatty acid. For example, the hexadecanoic acid ester of tricontanol is commonly reported to be a major component of beeswax. Other suitable waxes according to the invention include the synthetic waxes including polyethylene polyoxyethylene and hydrocarbon waxes derived from carbon monoxide and hydrogen.

Representative waxes also include: cerosin; cetyl esters; hydrogenated joioba oil; hydrogenated jojoba wax; hydrogenated rice bran wax; Japan wax; jojoba butter; jojoba oil; jojoba wax; munk wax; montan acid wax; ouricury wax; rice bran wax; shellac wax; sufurized jojoba oil; synthetic beeswax; synthetic jojoba oils; trihydroxystearin; cetyl alcohol; stearyl alcohol; cocoa butter; fatty acids of lanolin; mono-, di- and 25 triglycerides which are solid at 25 DEG C., e.g., glyceyl tribehenate (a triester of behenic acid and glycerine) and C1g-C36 acid triglyceride (a mixture of triesters of C1g-C36 carboxylic acids and glycerine) available from Croda, Inc., New York, N.Y. under the tradenames Syncrowax HRC and Syncrowax HGL-C, respectively; fatty esters which are solid at 25 DEG C.; silicone waxes such as methyloctadecaneoxypolysiloxane and poly (dimethylsiloxy) stearoxysiloxane; stearyl mono- and diethanolamide; rosin and its derivatives such as the abietates of glycol and glycerol; hydrogenated oils solid at 25 DEG C.; and sucroglycerides. Thickeners (viscosity control agents) which may be used in effective amounts in aqueous systems include: algin; carbomers such as carbomer 934, 934P, 940 and 941; cellulose gum; cetearyl alcohol, cocamide DEA, dextrin; gelatin; hydroxyethylcellulose; hydroxypropylcellulose; hydroxypropyl methylcellulose; magnesium aluminum silicate; myristyl alcohol; oat flour; oleamide DEA; oleyl alcohol; PEG-7M; PEG-14M; PEG-9OM; stearamide DEA; stearamide MEA; stearyl alcohol; tragacanth gum; wheat starch; xanthan gum; and the likein the above list of thickeners, DEA is diethanolamine, and MEA is monoethanolamine. Thickeners (viscosity control agents) which may be used in effective amounts in nonaqueous systems include aluminum stearates; beeswax; candelilla wax; carnauba; ceresin; cetearyl alcohol; cetyl alcohol; cholesterol; hydrated silica; hydrogenated castor oil; hydrogenated cottonseed oil; hydrogenated soybean oil; hydrogenated tallow glyceride; hydrogenated vegetable oil; hydroxypropyl cellulose; lanolin alcohol; myristyl alcohol; octytdodecyl stearoyl sulfate; oleyl alcohol; ozokerite; microcystalline wax; paraffin, pentaerythrityl tetraoctanoate; polyacrylamide; polybutene; polyethylene; propylene glycol dicaprylate; propylene glycol dipelargonate; stearalkonium hectorite; stearyl alcohol; stearyl stearate; synthetic beeswax; trihydroxystearin; trilinolein; tristearin; zinc stearate; and the like.

Customary native and synthetic thickeners or gel formers in formulations are crosslinked polyacrylic acids and derivatives thereof, polysaccharides, such as xanthane gum or alginates, carboxymethylcellulose or hydroxycarboxymethylcellulose, hydrocolloids such as gum Arabic or montmorillonite minerals, such as bentonites or fatty alcohols, polyvinyl alcohol and polyvinlypyrrolidone.

Other ingredients which can be added or used in a cosmetic or pharmaceutical composition according to the invention in amounts effective for their intended use, include: biological additives to enhance performance or consumer appeal such as amino acids, proteins, vanilla, aloe extract, bioflavinoids, and the like; buffering agents,; emulsion stabilizers; pH adjusters; opacifying agents; and propellants such as butane carbon clioxide, ethane, hydrochlorofluorocarbons 22 and 142b, hydrofluorocarbon 152a, isobutane, isopentane, nitrogen, nitrous oxide, pentane, propane, and the like.

Furthermore, the preparations according to the invention may also comprise compounds which have an antioxidative, free-radical scavenger, skin moisturizing or moisture-retaining, antierythematous, antiinflammatory or antiallergic action, in order to supplement or enhance their action. In particular, these compounds can be chosen from the group of vitamins, plant extracts, alpha- and beta-hydroxy acids, ceramides, antiinflammatory, antimicrobial or UV-filtering substances, and derivatives thereof and mixtures thereof. Advantageously, preparations according to the invention can also comprise substances which absorb UV radiation in the UV-B and/or UV-A region. The lipid phase is advantageously chosen from the group of substances of mineral oils, mineral waxes, branched and/or unbranched hydrocarbons and hydrocarbon waxes, triglycerides of saturated and/or unsaturated, branched and/or unbranched C.sub.8-C.sub.24-alkanecarboxylic acids; they can be chosen from synthetic, semisynthetic or natural oils, such as olive oil, palm oil, almond oil or mixtures; oils, fats or waxes, esters of saturated and/or unsaturated, branched and/or unbranched C.sub.3-C.sub.30-alkane carboxylic acids and saturated and/or unsaturated, branched and/or unbranched C.sub.3-C.sub.30-alcohols, from aromatic carboxylic acids and saturated and/or unsaturated, branched and/or unbranched C.sub.3-C.sub.30-alcohols, for example isopropyl myristate, isopropyl stearate, hexyldecyl stearate, oleyl oleate; and also synthetic, semisynthetic and natural mixtures of such esters, such as jojoba oil, alkyl benzoates or silicone oils, such as, for example, cyclomethicone, dimethylpolysiloxane, diethylpolysiloxane, octamethylcyclo-tetrasiloxane and mixtures thereof or dialkyl ethers.

The active ingredients according to the invention may, for example, be used in cosmetic compositions for the cleansing of the skin, such as bar soaps, toilet soaps, curd soaps, transparent soaps, luxury soaps, deodorizing soaps, cream soaps, baby soaps, skin protection soaps, abrasive soaps, syndets, liquid soaps, pasty soaps, soft soaps, washing pastes, liquid washing, showering and bath preparations, e.g. washing lotions, shower preparations, shower gels, foam baths, cream foam baths, oil baths, bath extracts, scrub preparations, in-situ products, shaving foams, shaving lotions, shaving creams. In addition, they are suitable for skin cosmetic preparations, such as W/O or O/W, skin and body creams, day and night creams, light protection compositions, aftersun products, multiple emulsions, gelees, microemulsions, liposome preparations, niosome preparations, lipogels, sportgels, moisturizing creams, bleaching creams, vitamin creams, skin lotions, care lotions, ampoules, preshaves, humectant lotions, cellulite creams, depigmentation compositions, massage preparations, body powders, deodorants, antiperspirants, repellents and others. The term “active ingredient” refers, for example, to the microorganism according to the present invention, mutant, derivative, inactive form, lysate, fraction or extract thereof as described above. Preferably, the term “active ingredient” as used in the compositions herein below is a substitute of, e.g., the microorganisms, mutants, derivatives, inactive forms, lysates, fractions or extracts thereof which are described herein above. If not indicated otherwise, the term “active ingredient” as used in the compositions described below refers to the percentage of, e.g., the microorganism according to the present invention, mutant, derivative, inactive form, lysate, fraction or extract thereof as described above, in the composition. Preferably, the term “active ingredient” refers to a microorganism according to the invention, e.g. Lactobacillus spec. or Leuconostoc spec. as defined herein above, in a concentration of e.g. 10²-10¹³ cells per ml. More preferably, the term “active ingredient” refers to a solution, e.g. an aqueous solution or any other suitable solution known to the person skilled in the art, comprising up to 0.001% to up to 99,999% of a microorganism according to the invention, e.g. Lactobacillus spec. or Leuconostoc spec. as defined herein above, in any suitable concentration known to the skilled person, e.g., a concentration of. 10²-10¹³ cells per ml. Even more preferably, the term refers to a solution comprising up to 0.001%, 0.01%, 0.1%, 1%, 5%, 10%, 15%, 20%, 25%, 30%, 40%, 50%, 60%, 70%, 80%, 90%, 95%, 99%, 99.9%, 99.99% or 99.999%, most preferably comprising up to 0.001 to up to 5%, of a microorganism according to the invention, e.g. Lactobacillus spec. or Leuconostoc spec., as defined herein above, in any suitable concentration known to the skilled person, e.g. a concentration of. 10²-10¹³ cells per ml.

In a preferred embodiment, a cosmetic composition comprises a daily care O/W formulation, which may contain, for example, the following ingredients in % in accordance with the International Nomenclature of Cosmetic Ingredients, INCI:

Active ingredient 1%:

| | | |
|---|---|---|
| A | 1.7 | ceteareth-6, stearyl alcohol |
| | 0.7 | ceteareth-25 |
| | 2.0 | diethylamino hydroxybenzoyl hexyl benzoate |
| | 2.0 | PEG-14 dimethicone |
| | 3.6 | cetearyl alcohol |
| | 6.0 | ethylhexyl methoxycinnamate |
| | 2.0 | dibutyl adipate |
| B | 5.0 | glycerol |
| | 0.2 | disodium EDTA |
| | 1.0 | panthenol |
| | q.s. | preservative |
| | 68.4 | aqua dem. |
| C | 4.0 | caprylic/capric triglyceride, sodium acrylates copolymer |
| D | 0.2 | sodium ascorbyl phosphate |
| | 1.0 | tocopheryl acetate |
| | 0.2 | bisabolol |
| | 1.0 | caprylic/capric triglyceride, sodium ascorbate, tocopherol, |
| | | retinol |
| | 1.0 | active ingredient |
| E | q.s. | sodium hydroxide |

### Active Ingredient 5%:

| | | |
|---|---|---|
| A | 1.7 | ceteareth-6, stearyl alcohol |
| | 0.7 | ceteareth-25 |
| | 2.0 | diethylamino hydroxybenzoyl hexyl benzoate |
| | 2.0 | PEG-14 dimethicone |
| | 3.6 | cetearyl alcohol |
| | 6.0 | ethylhexyl methoxycinnamate |
| | 2.0 | dibutyl adipate |
| B | 5.0 | glycerol |
| | 0.2 | disodium EDTA |
| | 1.0 | panthenol |
| | q.s. | preservative |
| | 64.4 | aqua dem. |
| C | 4.0 | caprylic/capric triglyceride, sodium acrylates copolymer |
| D | 0.2 | sodium ascorbyl phosphate |
| | 1.0 | tocopheryl acetate |
| | 0.2 | bisabolol |
| | 1.0 | caprylic/capric triglyceride, sodium ascorbate, tocopherol, |
| | | retinol |
| | 5.0 | active ingredient |
| E | q.s. | sodium hydroxide |

Phases A and B are separately heated to app. 80° C. Phase B is subsequently stirred into phase A and homogenized. Phase C is stirred into a combination of phases A and B and homogenized. The mixture is under agitation cooled down to app. 40° C.; then phase D is added and the pH is adjusted with phase E to approx. 6.5. The solution is subsequently homogenized and cooled down to room temperature.

In a further preferred embodiment, a cosmetic composition comprises a protecting day cream O/W formulation, which may contain, for example, the following ingredients in % in accordance with the International Nomenclature of Cosmetic Ingredients, INCI:

### Active Ingredient 1%:

| | | |
|---|---|---|
| A | 1.7 | ceteareth-6, stearyl alcohol |
| | 0.7 | ceteareth-25 |
| | 2.0 | diethylamino hydroxybenzoyl hexyl benzoate |
| | 2.0 | PEG-14 dimethicone |
| | 3.6 | cetearyl alcohol |
| | 6.0 | ethylhexyl methoxycinnamate |
| | 2.0 | dibutyl adipate |
| B | 5.0 | glycerol |
| | 0.2 | disodium EDTA |
| | 1.0 | panthenol |
| | q.s. | preservative |
| | 68.6 | aqua dem. |
| C | 4.0 | caprylic/capric triglyceride, sodium acrylates copolymer |
| D | 1.0 | sodium ascorbyl phosphate |
| | 1.0 | tocopheryl acetate |
| | 0.2 | bisabolol |
| | 1.0 | active ingredient |
| E | q.s. | sodium hydroxide |

### Active Ingredient 5%:

| | | |
|---|---|---|
| A | 1.7 | ceteareth-6, stearyl alcohol |
| | 0.7 | ceteareth-25 |
| | 2.0 | diethylamino hydroxybenzoyl hexyl benzoate |
| | 2.0 | PEG-14 dimethicone |
| | 3.6 | cetearyl alcohol |
| | 6.0 | ethylhexyl methoxycinnamate |
| | 2.0 | dibutyl adipate |
| B | 5.0 | glycerol |
| | 0.2 | disodium EDTA |
| | 1.0 | panthenol |
| | q.s. | preservative |
| | 64.6 | aqua dem. |
| C | 4.0 | caprylic/capric triglyceride, sodium acrylates copolymer |
| D | 1.0 | sodium ascorbyl phosphate |
| | 1.0 | tocopheryl acetate |
| | 0.2 | bisabolol |
| | 5.0 | active ingredient |
| E | q.s. | sodium hydroxide |

Phases A and B are separately heated to app. 80° C. Phase B is subsequently stirred into phase A and homogenized. Phase C is introduced into a combination of phases A and B and homogenized. The mixture is under agitation cooled down to app. 40° C.; then phase D is added and the pH is adjusted with phase E to about 6.5. The solution is subsequently homogenized and cooled down to room temperature.

In a further preferred embodiment, a cosmetic composition comprises a skin cleanser O/W formulation, which may contain, for example, the following ingredients in % in accordance with the International Nomenclature of Cosmetic Ingredients, INCI:

### Active Ingredient 1%:

| | | |
|---|---|---|
| A | 10.0 | cetearyl ethylhexanoate |
| | 10.0 | caprylic/capric triglyceride |
| | 1.5 | cyclopentasiloxane, cyclohexasilosane |
| | 2.0 | PEG-40 hydrogenated castor oil |
| B | 3.5 | caprylic/capric triglyceride, sodium acrylates copolymer |
| C | 1.0 | tocopheryl acetate |
| | 0.2 | bisabolol |
| | q.s. | preservative |
| | q.s. | perfume oil |
| D | 3.0 | polyquaternium-44 |
| | 0.5 | cocotrimonium methosulfate |
| | 0.5 | ceteareth-25 |
| | 2.0 | panthenol, propylene glycol |
| | 4.0 | propylene glycol |
| | 0.1 | disodium EDTA |
| | 1.0 | active ingredient |
| | 60.7 | aqua dem. |

### Active Ingredient 5%:

| | | |
|---|---|---|
| A | 10.0 | cetearyl ethylhexanoate |
| | 10.0 | caprylic/capric triglyceride |
| | 1.5 | cyclopentasiloxane, cyclohexasilosane |
| | 2.0 | PEG-40 hydrogenated castor oil |
| B | 3.5 | caprylic/capric triglyceride, sodium acrylates copolymer |
| C | 1.0 | tocopheryl acetate |
| | 0.2 | bisabolol |
| | q.s. | preservative |
| | q.s. | perfume oil |
| D | 3.0 | polyquaternium-44 |
| | 0.5 | cocotrimonium methosulfate |
| | 0.5 | ceteareth-25 |
| | 2.0 | panthenol, propylene glycol |
| | 4.0 | propylene glycol |
| | 0.1 | disodium EDTA |
| | 5.0 | active ingredient |
| | 56.7 | aqua dem. |

Initially, phase A is dissolved and phase B subsequently stirred into phase A. Subsequently, phase C is introduced into the combination of phases A and B. In a next step, phase D is dissolved and stirred into combined phases A, B and C. The mixture is homogenized and stirred for 15 min.

In a further preferred embodiment, a cosmetic composition comprises a daily care body spray formulation, which may contain, for example, the following ingredients in % in accordance with the International Nomenclature of Cosmetic Ingredients, INCI:

### Active Ingredient 1%:

| | | |
|---|---|---|
| A | 3.0 | ethylhexyl methoxycinnamate |
| | 2.0 | diethylamino hydroxybenzoyl hexyl benzoate |
| | 1.0 | polyquaternium-44 |
| | 3.0 | propylene glycol |
| | 2.0 | panthenol, propylene glycol |
| | 1.0 | cyclopentasiloxane, cyclohexasiloxane |
| | 10.0 | octyldodecanol |
| | 0.5 | PVP |
| | 10.0 | caprylic/capric triglyceride |
| | 3.0 | C12-15 alkyl benzoate |
| | 3.0 | glycerol |
| | 1.0 | tocopheryl acetate |
| | 0.3 | bisabolol |
| | 1.0 | active ingredient |
| | 59.2 | alcohol |

### Active Ingredient 5%:

| | | |
|---|---|---|
| A | 3.0 | ethylhexyl methoxycinnamate |
| | 2.0 | diethylamino hydroxybenzoyl hexyl benzoate |
| | 1.0 | polyquaternium-44 |
| | 3.0 | propylene glycol |
| | 2.0 | panthenol, propylene glycol |
| | 1.0 | cyclopentasiloxane, cyclohexasiloxane |
| | 10.0 | octyldodecanol |
| | 0.5 | PVP |
| | 10.0 | caprylic/capric triglyceride |
| | 3.0 | C12-15 alkyl benzoate |
| | 3.0 | glycerol |
| | 1.0 | tocopheryl acetate |
| | 0.3 | bisabolol |
| | 5.0 | active ingredient |
| | 55.2 | alcohol |

The components of phase A are weighed out and dissolved until clearness.

In a further preferred embodiment, a cosmetic composition comprises a skin gel, which may contain, for example, the following ingredients in % in accordance with the International Nomenclature of Cosmetic Ingredients, INCI:

### Active Ingredient 1%:

| | | |
|---|---|---|
| | 3.6 | PEG-40 hydrogenated castor oil |
| | 15.0 | alcohol |
| | 0.1 | bisabolol |
| | 0.5 | tocopheryl acetate |
| | q.s. | perfume oil |
| B | 3.0 | panthenol |
| | 0.6 | carbomer |
| | 1.0 | active ingredient |
| | 75.4 | aqua dem. |
| C | 0.8 | triethanolamine |

### Active Ingredient 5%:

| | | |
|---|---|---|
| | 3.6 | PEG-40 hydrogenated castor oil |
| | 15.0 | alcohol |
| | 0.1 | bisabolol |
| | 0.5 | tocopheryl acetate |
| | q.s. | perfume oil |
| B | 3.0 | panthenol |
| | 0.6 | carbomer |
| | 5.0 | active ingredient |
| | 71.4 | aqua dem. |
| C | 0.8 | triethanolamine |

Initially, phase A is dissolved until clearness. Phase B is macerated and subsequently neutralized with phase C. In a next step, phase A is stirred into the homogenized phase B and the mixture is homogenized.

In yet a further preferred embodiment, a cosmetic composition comprises an after shave lotion, which may contain, for example, the following ingredients in % in accordance with the International Nomenclature of Cosmetic Ingredients, INCI:

### Active Ingredient 1%:

| | | |
|---|---|---|
| A | 10.0 | cetearyl ethylhexanoate |
| | 5.0 | tocopheryl acetate |
| | 1.0 | bisabolol |
| | 0.1 | perfume oil |
| | 0.3 | acrylates/c10-30 alkyl acrylate crosspolymer |
| B | 15.0 | alcohol |
| | 1.0 | panthenol |
| | 3.0 | glycerol |
| | 1.0 | active ingredient |
| | 0.1 | triethanolamine |
| | 63.5 | aqua dem. |

### Active Ingredient 5%:

| | | |
|---|---|---|
| A | 10.0 | cetearyl ethylhexanoate |
| | 5.0 | tocopheryl acetate |
| | 1.0 | bisabolol |
| | 0.1 | perfume oil |
| | 0.3 | acrylates/c10-30 alkyl acrylate crosspolymer |
| B | 15.0 | alcohol |
| | 1.0 | panthenol |
| | 3.0 | glycerol |
| | 5.0 | active ingredient |
| | 0.1 | triethanolamine |
| | 59.5 | aqua dem. |

The component of phase A are mixed. In a next step, phase B is dissolved and introduced into phase A and subsequently homogenized.

In a further preferred embodiment, a cosmetic composition comprises an antitranspiration roll-on, which may contain, for example, the following ingredients in % in accordance with the International Nomenclature of Cosmetic Ingredients, INCI:

### Active Ingredient 1%:

| | | |
|---|---|---|
| A | 0.40 | hydroxyethylcellulose |
| | 50.0 | aqua dem. |
| B | 25.0 | alcohol |
| | 0.1 | bisabolol |
| | 0.3 | farnesol |
| | 2.0 | PEG-40 hydrogenated castor oil q.s. perfume oil |
| C | 3.0 | dipropylene glycol |
| | 3.0 | PEG-14 demethicone |
| | 3.0 | polyquaternium-16 |
| | 8.2 | aqua dem. |
| D | 1.0 | active ingredient |

### Active Ingredient 5%:

| | | |
|---|---|---|
| A | 0.40 | hydroxyethylcellulose |
| | 46.0 | aqua dem. |
| B | 25.0 | alcohol |
| | 0.1 | bisabolol |
| | 0.3 | farnesol |
| | 2.0 | PEG-40 hydrogenated castor oil q.s. perfume oil |
| C | 3.0 | dipropylene glycol |
| | 3.0 | PEG-14 demethicone |
| | 3.0 | polyquaternium-16 |
| | 8.2 | aqua dem. |
| D | 5.0 | active ingredient |

Phase A is swollen, phases B and C are solubilized independently. Subsequently, phases B and A are stirred into phase C. Finally, phase D is added.

In a further preferred embodiment, a cosmetic composition comprises a transparent deo stick, which may contain, for example, the following ingredients in % in accordance with the International Nomenclature of Cosmetic Ingredients, INCI:

### Active Ingredient 1%:

| | | |
|---|---|---|
| A | 3.0 | ceteareth-25 |
| | 3.0 | PEG-40 hydrogenated castor oil |
| | 0.2 | bisabolol rac. |
| | 1.0 | tocopheryl acetate |
| | 3.0 | perfume oil |
| | 5.0 | sodium stearate |
| | 15.0 | glycerol 87% |
| | 60.0 | propylene glycol |
| | 9.3 | aqua dem. |
| B | 1.0 | active ingredient |

### Active Ingredient 5%:

| | | |
|---|---|---|
| A | 3.0 | ceteareth-25 |
| | 3.0 | PEG-40 hydrogenated castor oil |
| | 0.2 | bisabolol rac. |
| | 1.0 | tocopheryl acetate |
| | 3.0 | perfume oil |
| | 5.0 | sodium stearate |
| | 15.0 | glycerol 87% |
| | 60.0 | propylene glycol |
| | 5.3 | aqua dem. |
| B | 5.0 | active ingredient |

Components of phase A are weighed out and melted. Subsequently, phase B is added.

In a further preferred embodiment, a cosmetic composition comprises an antitranspiration spray, which may contain, for example, the following ingredients in % in accordance with the International Nomenclature of Cosmetic Ingredients, INCI:

### Active Ingredient 1%:

| | | |
|---|---|---|
| A | 3.0 | PEG-40 hydrogenated castor oil |
| | 0.2 | phytantriol |
| | 0.5 | perfume oil |
| | 40.0 | alcohol |
| B | 53.49 | aqua dem. |
| | 2.0 | propylene glycol |
| | 0.5 | panthenol |
| | 0.01 | BHT |
| C | 1.0 | active ingredient |

### Active Ingredient 5%:

| | | |
|---|---|---|
| A | 3.0 | PEG-40 hydrogenated castor oil |
| | 0.2 | phytantriol |
| | 0.5 | perfume oil |
| | 40.0 | alcohol |
| B | 49.49 | aqua dem. |
| | 2.0 | propylene glycol |
| | 0.5 | panthenol |
| | 0.01 | BHT |
| C | 5.0 | active ingredient |

Phase A is solubilized. In a next step the components of phase B added successively. Finally, phase C is added.

In a further preferred embodiment, a cosmetic composition comprises a deo-stick, which may contain, for example, the following ingredients in % in accordance with the International Nomenclature of Cosmetic Ingredients, INCI:

### Active Ingredient 1%:

| | | |
|---|---|---|
| A | 26.0 | stearyl alcohol |
| | 60.0 | cyclopentasiloxane, cyclohexasiloxane |
| | 5.0 | PEG-40 hydrogenated castor oil |
| | 2.5 | isopropyl palmitate |
| B | 1.44 | perfume oil |
| | 0.05 | BHT |
| C | 1.0 | active ingredient |

### Active Ingredient 5%:

| | | |
|---|---|---|
| A | 26.0 | stearyl alcohol |
| | 56.0 | cyclopentasiloxane, cyclohexasiloxane |
| | 5.0 | PEG-40 hydrogenated castor oil |
| | 2.5 | isopropyl palmitate |
| B | 1.44 | perfume oil |
| | 0.05 | BHT |
| C | 5.0 | active ingredient |

The components of phase A are weighed out and melted. Phase A is subsequently cooled down while stirring to about 50° C. The components of phase B and C are homogenized and added successively.

In a further preferred embodiment, a cosmetic composition comprises a transparent deo-roll on, which may contain, for example, the following ingredients in % in accordance with the International Nomenclature of Cosmetic Ingredients, INCI:

### Active Ingredient 1%:

| | | |
|---|---|---|
| A | 0.40 | hydroxyethylcellulose |
| | 50.0 | aqua dem. |
| B | 2.0 | PEG-40 hydrogenated castor oil |
| | 0.1 | bisabolol |
| | 0.3 | farnesol |
| | 0.5 | perfume oil |
| | 7.6 | aqua dem. |
| | 25.0 | alcohol |
| C | 3.0 | propylene glycol |
| | 3.0 | PEG-14 demethicone |
| | 3.0 | polyquaternium-16 |
| | 0.1 | allantoin |
| D | 1.0 | active ingredient |

### Active Ingredient 5%:

| | | |
|---|---|---|
| A | 0.40 | hydroxyethylcellulose |
| | 46.0 | aqua dem. |
| B | 2.0 | PEG-40 hydrogenated castor oil |
| | 0.1 | bisabolol |
| | 0.3 | farnesol |
| | 0.5 | perfume oil |
| | 7.6 | aqua dem. |
| | 25.0 | alcohol |
| C | 3.0 | propylene glycol |
| | 3.0 | PEG-14 demethicone |
| | 3.0 | polyquaternium-16 |
| | 0.1 | allantoin |
| D | 5.0 | active ingredient |

Phase A is swollen, phase B is solubilized. Subsequently, phase C is added and stirred. Finally, phases B, C and D are stirred into phase A.

In a further preferred embodiment, a cosmetic composition comprises an emulsion, which may contain, for example, the following ingredients in % in accordance with the International Nomenclature of Cosmetic Ingredients, INCI:

### Active Ingredient 1%:

| | | |
|---|---|---|
| A | 1.5 | ceteareth-6, stearyl alcohol |
| | 2.0 | ceteareth-25 |
| | 5.0 | PEG-40 hydrogenated castor oil |
| | 1.5 | glyceryl stearate |
| | 1.0 | cetearyl alcohol |
| | 0.5 | Eucerinum anhydricum |
| | 0.2 | phytantriol |
| | 1.0 | cetyl palpitate |
| | 5.0 | dicaprylyl ether |
| | 0.3 | farnesol |
| B | q.s. | preservative |
| | 72.0 | aqua dem. |
| C | q.s. | perfume oil |
| D | 1.0 | active ingredient |

### Active Ingredient 5%:

| | | |
|---|---|---|
| A | 1.5 | ceteareth-6, stearyl alcohol |
| | 2.0 | ceteareth-25 |
| | 5.0 | PEG-40 hydrogenated castor oil |
| | 1.5 | glyceryl stearate |
| | 1.0 | cetearyl alcohol |
| | 0.5 | Eucerinum anhydricum |
| | 0.2 | phytantriol |
| | 1.0 | cetyl palpitate |
| | 5.0 | dicaprylyl ether |
| | 0.3 | farnesol |
| B | q.s. | preservative |
| | 68.0 | aqua dem. |
| C | q.s. | perfume oil |
| D | 5.0 | active ingredient |

Phases A and B are heated separately to approx. 80° C. Phase B is stirred into phase A and homogenized for 3 minutes. Subsequently, the mixture is cooled down to 40° C. and phases C and D are added. Finally, the mixture is stirred and cooled down to room temperature.

In a further preferred embodiment, a cosmetic composition comprises a deo-pump spray, which may contain, for example, the following ingredients in % in accordance with the International Nomenclature of Cosmetic Ingredients, INCI:

### Active Ingredient 1%:

| | | |
|---|---|---|
| A | 5.0 | PEG-40 hydrogenated castor oil |
| | 0.3 | PEG-7 hydrogenated castor oil |
| | 1.0 | glyceryl stearate |
| | 1.0 | cetearyl alcohol |
| | 5.0 | cyclopentasiloxane |
| | 0.5 | Eucerinum anhydricum |
| | 0.2 | phytantriol |
| | 5.0 | dicaprylyl ether |
| | 0.3 | farnesol |
| B | q.s. | preservative |
| | 76.7 | aqua dem. |
| C | q.s. | perfume oil |
| D | 1.0 | active ingredient |

### Active Ingredient 5%:

| | | |
|---|---|---|
| A | 5.0 | PEG-40 hydrogenated castor oil |
| | 0.3 | PEG-7 hydrogenated castor oil |
| | 1.0 | glyceryl stearate |
| | 1.0 | cetearyl alcohol |
| | 6.0 | cyclopentasiloxane |
| | 0.5 | Eucerinum anhydricum |
| | 0.2 | phytantriol |
| | 5.0 | dicaprylyl ether |
| | 0.3 | farnesol |
| B | q.s. | preservative |
| | 72.7 | aqua dem. |
| C | q.s. | perfume oil |
| D | 5.0 | active ingredient |

Phases A and B are heated separately to approx. 80° C. Phase B is homogenized and stirred into phases A and C. Subsequently, the mixture is cooled down to 40° C. and phase D is added. Finally, the mixture is stirred and cooled down to room temperature.

In a further preferred embodiment, a cosmetic composition comprises a deo-lotion, which may contain, for example, the following ingredients in % in accordance with the International Nomenclature of Cosmetic Ingredients, INCI:

### Active Ingredient 1%:

| | | |
|---|---|---|
| A | 1.5 | ceteareth-6, stearyl alcohol |
| | 1.5 | ceteareth-25 |
| | 2.0 | PEG-40 hydrogenated castor oil |
| | 2.0 | glyceryl stearate |
| | 2.0 | cetearyl alcohol |
| | 2.0 | cetyl alcohol |
| | 2.0 | hydrogenated coco-glycerides |
| | 8.0 | decyl oleate |
| | 0.5 | PEG-14 demehicone |
| | 0.3 | farnesol |
| B | q.s. | preservative |
| | 75.2 | aqua dem. |
| C | q.s. | perfume oil |
| D | 1.0 | active ingredient |

### Active Ingredient 5%:

| | | |
|---|---|---|
| A | 1.5 | ceteareth-6, stearyl alcohol |
| | 1.5 | ceteareth-25 |
| | 2.0 | PEG-40 hydrogenated castor oil |
| | 2.0 | glyceryl stearate |
| | 2.0 | cetearyl alcohol |
| | 2.0 | cetyl alcohol |
| | 2.0 | hydrogenated coco-glycerides |
| | 8.0 | decyl oleate |
| | 0.5 | PEG-14 demehicone |
| | 0.3 | farnesol |
| B | q.s. | preservative |
| | 71.2 | aqua dem. |
| C | q.s. | perfume oil |
| D | 5.0 | active ingredient |

Phases A and B are heated separately to approx. 80° C. Phase B is homogenized and stirred into phase A. Subsequently, the mixture is cooled down to 40° C. and phases C and D are added. Finally, the mixture is stirred and cooled down to room temperature.

In a further preferred embodiment, a cosmetic composition comprises a deo-lotion, type O/W which may contain, for example, the following ingredients in % in accordance with the International Nomenclature of Cosmetic Ingredients, INCI:

### Active Ingredient 1%:

| | | |
|---|---|---|
| A | 2.0 | ceteareth-6, stearyl alcohol |
| | 2.0 | ceteareth-25 |
| | 4.0 | cetearyl ethylhexanoate |
| | 2.0 | cetearyl alcohol |
| | 2.0 | hydrogenated coco-glycerides |
| | 1.0 | glyceryl stearate |
| | 1.0 | mineral oil |
| | 0.5 | dimethicone |
| | 0.2 | bisabolol |
| B | 2.0 | panthenol, propylene glycol |
| | 2.0 | propylene glycol |
| | q.s. | preservative |
| | 78.9 | aqua dem. |
| C | 1.2 | caprylic/capric triglyceride, sodium acrylates copolymer |
| D | 0.2 | tocopherol |
| | q.s. | perfume oil |
| E | 1.0 | active ingredient |

### Active Ingredient 5%:

| | | |
|---|---|---|
| A | 2.0 | ceteareth-6, stearyl alcohol |
| | 2.0 | ceteareth-25 |
| | 4.0 | cetearyl ethylhexanoate |
| | 2.0 | cetearyl alcohol |
| | 2.0 | hydrogenated coco-glycerides |
| | 1.0 | glyceryl stearate |
| | 1.0 | mineral oil |
| | 0.5 | dimethicone |
| | 0.2 | bisabolol |
| B | 2.0 | panthenol, propylene glycol |
| | 2.0 | propylene glycol |
| | q.s. | preservative |
| | 74.9 | aqua dem. |
| C | 1.2 | caprylic/capric triglyceride, sodium acrylates copolymer |
| D | 0.2 | tocopherol |
| | q.s. | perfume oil |
| E | 5.0 | active ingredient |

In a further preferred embodiment, a cosmetic composition comprises a foot balsam, which may contain, for example, the following ingredients in % in accordance with the International Nomenclature of Cosmetic Ingredients, INCI:

### Active Ingredient 1%:

| | | |
|---|---|---|
| A | 2.0 | ceteareth-6, stearyl alcohol |
| | 2.0 | ceteareth-25 |
| | 5.0 | cetearyl ethylhexanoate |
| | 4.0 | cetyl alcohol |
| | 4.0 | glyceryl stearate |
| | 5.0 | mineral oil |
| | 0.2 | menthol |
| | 0.5 | camphor |
| B | 69.3 | aqua dem. |
| | q.s. | preservative |
| C | 1.0 | bisabolol |
| | 1.0 | tocopheryl acetate |
| D | 1.0 | active ingredient |
| | 5.0 | witch hazel extract |

### Active Ingredient 5%:

| | | |
|---|---|---|
| A | 2.0 | ceteareth-6, stearyl alcohol |
| | 2.0 | ceteareth-25 |
| | 5.0 | cetearyl ethylhexanoate |
| | 4.0 | cetyl alcohol |
| | 4.0 | glyceryl stearate |
| | 5.0 | mineral oil |
| | 0.2 | menthol |
| | 0.5 | camphor |
| B | 65.3 | aqua dem. |
| | q.s. | preservative |
| C | 1.0 | bisabolol |
| | 1.0 | tocopheryl acetate |
| D | 5.0 | active ingredient |
| | 5.0 | witch hazel extract |

Phases A and B are separately heated to app. 80° C. Phase B is subsequently stirred into phase A and homogenized. The mixture is under agitation cooled down to app. 40° C.; then phases C and D are added. Subsequently, the mixture is homogenized and cooled down to room temperature under agitation.

In a further preferred embodiment, a cosmetic composition comprises a W/O emulsion with bisabolol, which may contain, for example, the following ingredients in % in accordance with the International Nomenclature of Cosmetic Ingredients, INCI:

### Active Ingredient 1%:

| | | |
|---|---|---|
| A | 6.0 | PEG-7 hydrogenated castor oil |
| | 8.0 | cetearyl ethylhexanoate |
| | 5.0 | isopropyl myristate |
| | 15.0 | mineral oil |
| | 0.3 | magnesium stearate |
| | 0.3 | aluminum stearate |
| | 2.0 | PEG-45/dodecyl glycol copolymer |
| B | 5.0 | glycerol |
| | 0.7 | magnesium sulfate |
| | 55.6 | aqua dem. |
| C | 1.0 | active ingredient |
| | 0.5 | tocopheryl acetate |
| | 0.6 | bisabolol |

### Active Ingredient 5%:

| | | |
|---|---|---|
| A | 6.0 | PEG-7 hydrogenated castor oil |
| | 8.0 | cetearyl ethylhexanoate |
| | 5.0 | isopropyl myristate |
| | 15.0 | mineral oil |
| | 0.3 | magnesium stearate |
| | 0.3 | aluminum stearate |
| | 2.0 | PEG-45/dodecyl glycol copolymer |
| B | 5.0 | glycerol |
| | 0.7 | magnesium sulfate |
| | 51.6 | aqua dem. |
| C | 5.0 | active ingredient |
| | 0.5 | tocopheryl acetate |
| | 0.6 | bisabolol |

Phases A and B are separately heated to app. 85° C. Phase B is subsequently stirred into phase A and homogenized. The mixture is under agitation cooled down to app. 40° C.; then phase C is added. Subsequently, the mixture is shortly homogenized and cooled down to room temperature under agitation.

In a further preferred embodiment, a cosmetic composition comprises a shower gel, which may contain, for example, the following ingredients in % in accordance with the International Nomenclature of Cosmetic Ingredients, INCI:

### Active Ingredient 1%:

| | | |
|---|---|---|
| A | 40.0 | sodium laureth sulfate |
| | 5.0 | decyl glucoside |
| | 5.0 | cocamidopropyl betaine |
| | 1.0 | active ingredient |
| | 1.0 | panthenol |
| | q.s. | perfume oil |
| | q.s. | preservative |
| | 2.0 | sodium chloride |
| | 46.0 | aqua dem. |
| B | q.s. | citric acid |

### Active Ingredient 5%:

| | | |
|---|---|---|
| A | 40.0 | sodium laureth sulfate |
| | 5.0 | decyl glucoside |
| | 5.0 | cocamidopropyl betaine |
| | 5.0 | active ingredient |
| | 1.0 | panthenol |
| | q.s. | perfume oil |
| | q.s. | preservative |
| | 2.0 | sodium chloride |
| | 42.0 | aqua dem. |
| B | q.s. | citric acid |

The components of phase A are mixed and dissolved. The pH is adjusted to 6-7 with phase B, i.e. citric acid.

In a further preferred embodiment, a cosmetic composition comprises a moistening body care creme, which may contain, for example, the following ingredients in % in accordance with the International Nomenclature of Cosmetic Ingredients, INCI:

### Active Ingredient 1%:

| | | |
|---|---|---|
| A | 2.0 | ceteareth-25 |
| | 2.0 | ceteareth-6, stearyl alcohol |
| | 3.0 | cetearyl ethylhexanoate |
| | 1.0 | dimethicone |
| | 4.0 | cetearyl alcohol |
| | 3.0 | glyceryl stearate SE |
| | 5.0 | mineral oil |
| | 4.0 | Simmondsia chinensis (jojoba) seed oil |
| | 3.0 | mineral oil, lanolin alcohol |
| B | 5.0 | propylene glycol |
| | 1.0 | active ingredient |
| | 1.0 | panthenol |
| | 0.5 | magnesium aluminum silicate |
| | q.s | preservative |
| | 65.5 | aqua dem. |
| C | q.s. | perfume oil |
| D | q.s. | citric acid |

### Active Ingredient 5%:

| | | |
|---|---|---|
| A | 2.0 | ceteareth-25 |
| | 2.0 | ceteareth-6, stearyl alcohol |
| | 3.0 | cetearyl ethylhexanoate |
| | 1.0 | dimethicone |
| | 4.0 | cetearyl alcohol |
| | 3.0 | glyceryl stearate se |
| | 5.0 | mineral oil |
| | 4.0 | simmondsia chinensis (jojoba) seed oil |
| | 3.0 | mineral oil, lanolin alcohol |
| B | 5.0 | propylene glycol |
| | 5.0 | active ingredient |
| | 1.0 | panthenol |
| | 0.5 | magnesium aluminum silicate |
| | q.s | preservative |
| | 61.5 | aqua dem. |
| C | q.s. | perfume oil |
| D | q.s. | citric acid |

Phases A and B are separately heated to app. 80° C. Phase B is briefly pre-homogenized. Subsequently phase B is stirred into phase A and homogenized. The mixture is cooled down to app. 40° C.; then phase C is added. Subsequently, the mixture is well homogenized. The pH is adjusted to 6-7 with phase D, i.e. citric acid.

In a further preferred embodiment, a cosmetic composition comprises a moistening body care creme, which may contain, for example, the following ingredients in % in accordance with the International Nomenclature of Cosmetic Ingredients, INCI:

### Active Ingredient 1%:

| | | |
|---|---|---|
| A | 6.0 | PEG-7 hydrogenated castor oil |
| | 10.0 | cetearyl ethylhexanoate |
| | 5.0 | isopropyl myristate |
| | 7.0 | mineral oil |
| | 0.5 | shea butter (butyrospermum parkii) |
| | 0.5 | aluminum stearate |
| | 0.5 | magnesium stearate |
| | 0.2 | bisabolol |
| | 0.7 | quaternium-18-hectorite |
| B | 5.0 | dipropylene glycol |
| | 0.7 | magnesium sulfate |
| | q.s. | preservative |
| | 62.9 | aqua dem. |
| C | q.s. | perfume oil |
| | 1.0 | active ingredient |

### Active Ingredient 5%:

| | | |
|---|---|---|
| A | 6.0 | PEG-7 hydrogenated castor oil |
| | 10.0 | cetearyl ethylhexanoate |
| | 5.0 | isopropyl myristate |
| | 7.0 | mineral oil |
| | 0.5 | shea butter (butyrospermum parkii) |
| | 0.5 | aluminum stearate |
| | 0.5 | magnesium stearate |
| | 0.2 | bisabolol |
| | 0.7 | quaternium-18-hectorite |
| B | 5.0 | dipropylene glycol |
| | 0.7 | magnesium sulfate |
| | q.s. | preservative |
| | 58.9 | aqua dem. |
| C | q.s. | perfume oil |
| | 5.0 | active ingredient |

Phases A and B are separately heated to app. 80° C. Phase B is stirred into phase A and homogenized. The mixture is cooled down under agitation to app. 40° C.; then phase C is added. Subsequently, the mixture is homogenized. The mixture is cooled down to room temperature under agitation.

In a further preferred embodiment, a cosmetic composition comprises a foam O/W emulsion, which may contain, for example, the following ingredients in % in accordance with the International Nomenclature of Cosmetic Ingredients, INCI:

| | Example 1 | | Example 2 | |
|---|---|---|---|---|
| Ingredients (in %) | W.-% | Vol-% | W.-% | Vol-% |
| stearic acid | 5.00 | | 1.00 | |
| cetyl alcohol | 5.50 | | | |
| cetearyl alcohol | | | 2.00 | |
| PEG-40 stearate | 8.50 | | | |
| PEG-20 stearate | | | 1.00 | |
| caprylic/capric triglyceride | 4.00 | | 2.00 | |
| C12-15 alkyl benzoate | 10.00 | | 15.00 | |
| cyclomethicone | 4.00 | | | |
| dimethicone | | | 0.50 | |
| active ingredient | 5.0 | | 10.0 | |
| ethylhexyl isostearate | | | 5.00 | |
| myristyl myristate | | | 2.00 | |
| ceresin | 1.50 | | | |
| glycerol | | | 3.00 | |
| hydroxypropyl starch | 1.00 | | 3.50 | |
| phosphate | | | | |
| BHT | | | 0.02 | |
| disodium EDTA | 0.50 | | 0.10 | |
| perfume oil, preservative | q.s. | | q.s. | |
| colorant | q.s. | | q.s. | |
| potassium hydroxide | q.s. | | q.s. | |
| aqua dem. | ad 100 | | ad 100 | |
| | pH | | pH | |
| | adjusted | | adjusted | |
| | to 6.5-7.5 | | to 5.0-6.0 | |
| emulsion 1 | | 70 | | |
| emulsion 2 | | | | 35 |
| nitrogen | | 30 | | |
| propan/butan | | | | 65 |

In a further preferred embodiment, a cosmetic composition comprises a gel creme, which may contain, for example, the following ingredients in % in accordance with the International Nomenclature of Cosmetic Ingredients, INCI:

| Ingredients (in %) | Example 1 | Example 2 | Example 3 | Example 4 |
|---|---|---|---|---|
| acrylates/C10-30 alkylacrylate | 0.40 | 0.35 | 0.40 | 0.35 |
| crosspolymer | | | | |
| carbomer | 0.20 | 0.22 | 0.20 | 0.22 |
| xanthan gum | 0.10 | 0.13 | 0.10 | 0.13 |
| cetearyl alcohol | 3.00 | 2.50 | 3.00 | 2.50 |
| C12-15 alkyl benzoate | 4.00 | 4.50 | 4.00 | 4.50 |
| caprylic/capric triglyceride | 3.00 | 3.50 | 3.00 | 3.50 |
| Uvinul ® A Plus ™ | 2.00 | 1.50 | 0.75 | 1.00 |
| UvaSorb ® k2A Ethylhexyl Bis- | | 3.00 | | |
| Isopentylbenzoxazolylphenyl | | | | |
| Melamine | | | | |
| Uvinul ® MC 80 | 3.00 | | 1.00 | |
| bis-ethylhexyloxyphenol | | 1.50 | | 2.00 |
| methoxyphenyl triazine | | | | |
| butyl methoxydibenzoylmethane | | | 2.00 | |
| disodium phenyl dibenzimidazole | 2.50 | | 0.50 | 2.00 |
| tetrasulfonate | | | | |
| Uvinul ® T 150 | 4.00 | | 3.00 | 4.00 |
| octocrylene | | 4.00 | | |
| diethylhexyl butamido triazone | 1.00 | | | 2.00 |
| phenylbenzimidazole sulfonic acid | 0.50 | | 3.00 | |
| methylene bis-benzotriazolyl | 2.00 | | 0.50 | 1.50 |
| tetramethylbutylphenol | | | | |
| ethylhexyl salicylate | | | 3.00 | |
| drometrizole trisiloxane | | | 0.50 | |
| terephthaliden dicamphor sulfonic acid | | 1.50 | | 1.00 |
| diethylhexyl 2,6-naphthalate | 3.50 | 4.00 | 7.00 | 9.00 |
| titanium dioxide-microfine | 1.00 | | 3.00 | |
| zinc oxide-microfine | | | | 0.25 |
| active ingredient | 0.1 | 0.5 | 1.0 | 0.02 |
| cyclomethicone | 5.00 | 5.50 | 5.00 | 5.50 |
| dimethicone | 1.00 | 0.60 | 1.00 | 0.60 |
| glycerol | 1.00 | 1.20 | 1.00 | 1.20 |
| sodium hydroxide | q.s. | q.s. | q.s. | q.s. |
| preservative | 0.30 | 0.23 | 0.30 | 0.23 |
| perfume oil | 0.20 | | 0.20 | |
| aqua dem. | ad 100 | ad 100 | ad 100 | ad 100 |
| pH adjusted to 6.0 | | | | |

In a further preferred embodiment, a cosmetic composition comprises a hydrodispersion, which may contain, for example, the following ingredients in % in accordance with the International Nomenclature of Cosmetic Ingredients, INCI:

| Ingredients (in %) | Example 1 | Example 2 | Example 3 | Example 4 | Example 5 |
|---|---|---|---|---|---|
| ceteaereth-20 | 1.00 | | | 0.50 | |
| cetyl alcohol | | | 1.00 | | |
| sodium carbomer | | 0.20 | | 0.30 | |
| acrylates/C10-30 alkyl | 0.50 | | 0.40 | 0.10 | 0.50 |
| acrylate crosspolymer | | | | | |
| xanthan gum | | 0.30 | 0.15 | | |
| active ingredient | 5.0 | 0.5 | 3.0 | 0.1 | 10.0 |
| Uvinul ® A PLus ™ | 2.00 | 1.50 | 0.75 | 1.00 | 2.10 |
| UvaSorb ® k2A ethylhexyl | | 3.50 | | | |
| bis-isopentylbenzoxazolylphenyl | | | | | |
| melamine | | | | | |
| ethylhexyl | | | | | 5.00 |
| methoxycinnamate | | | | | |
| Uvinul ® MC 80 | | | | | |
| bis-ethylhexyloxyphenol | | 1.50 | | 2.00 | 2.50 |
| methoxyphenyl triazine | | | | | |
| butylmethoxy | | | 2.00 | | 2.00 |
| dibenzoylmethane | | | | | |
| dinatrium phenyl | 2.50 | | 0.50 | 2.00 | |
| dibenzimidazole | | | | | |
| tetrasulfonate | | | | | |
| ethyhexyl triazone | 4.00 | | 3.00 | 4.00 | |
| Uvinul ® T 150 | | | | | |
| octocrylene | | 4.00 | | | |
| diethylhexyl butamido | 1.00 | | | 2.00 | 1.00 |
| triazone | | | | | |
| phenylbenzimidazol | 0.50 | | 3.00 | | |
| sulfonic acid | | | | | |
| methylene bis- | 2.00 | | 0.50 | 1.50 | 2.50 |
| benzotriazolyl | | | | | |
| tetramethylbutylphenol | | | | | |
| ethylhexyl salicylate | | | 3.00 | | |
| drometrizol trisiloxane | | | 0.50 | | |
| terephthaliden dicamphor | | 1.50 | | 1.00 | 1.00 |
| sulfonic acid | | | | | |
| diethylhexyl 2,6- | | | 7.00 | | 9.00 |
| naphthalate | | | | | |
| titanium dioxide- | 1.00 | | 3.00 | | 3.50 |
| microfine | | | | | |
| zinc oxide-microfine | | | | 0.25 | |
| C12-15 alkyl benzoate | 2.00 | 2.50 | | | |
| dicapryl ether | | 4.00 | | | |
| butylenglycol | 4.00 | | 2.00 | 6.00 | |
| dicaprylate/dicaprate | | | | | |
| dicapryl carbonate | | 2.00 | 6.00 | | |
| dimethicone | | 0.50 | 1.00 | | |
| phenyl trimethicone | 2.00 | | 0.50 | | |
| butyrospermum parkii | | 2.00 | | 5.00 | |
| (shea butter) | | | | | |
| VP/hexadecene | 0.50 | | | 0.50 | 1.00 |
| copolymer | | | | | |
| tricontanyl PVP | 0.50 | | 1.00 | | |
| ethylhexylglycerol | | | 1.00 | | 0.80 |
| glycerol | 3.00 | 7.50 | | 7.50 | 8.50 |
| glycine soja (soybean) oil | | | 1.50 | | 1.00 |
| vitamin E acetate | 0.50 | | 0.25 | | 1.00 |
| glucosylrutin | 0.60 | | | 0.25 | |
| biosaccaride gum-1 | | 2.50 | 0.50 | | 2.00 |
| DMDM hydantoin | | 0.60 | 0.45 | 0.25 | |
| iodopropynyl | 0.20 | | | | |
| butylcarbamatec | | | | | |
| methylparaben | 0.50 | | 0.25 | 0.15 | |
| phenoxyethanol | 0.50 | 0.40 | | 1.00 | |
| trisodium EDTA | | 0.01 | 0.05 | | 0.10 |
| ethanol | 3.00 | 2.00 | 1.50 | | 7.00 |
| perfume oil | 0.20 | | 0.05 | 0.40 | |
| aqua dem. | ad 100 | ad 100 | ad 100 | ad 100 | ad 100 |

In a further preferred embodiment, a cosmetic composition comprises a stick, which may contain, for example, the following ingredients in % in accordance with the International Nomenclature of Cosmetic Ingredients, INCI:

| Ingredients (in %) | Example 1 | Example 2 | Example 3 | Example 4 |
|---|---|---|---|---|
| caprylic/capric | 12.00 | 10.00 | 6.00 | |
| triglyceride | | | | |
| octyldodecanol | 7.00 | 14.00 | 8.00 | 3.00 |
| butylene glycol | | | | 12.00 |
| dicaprylate/dicaprate | | | | |
| pentaerythrityl | 10.00 | 6.00 | 8.00 | 7.00 |
| tetraisostearate | | | | |
| polyglyceryl-3 | 2.50 | | | |
| diisostearate | | | | |
| bis-diglyceryl | 9.00 | 8.00 | 10.00 | 8.00 |
| polyacyladipate-2 | | | | |
| cetearyl alcohol | 8.00 | 11.00 | 9.00 | 7.00 |
| myristyl myristate | 3.50 | 3.00 | 4.00 | 3.00 |
| beeswax | 5.00 | 5.00 | 6.00 | 6.00 |
| copernicia | 1.50 | 2.00 | 2.00 | 1.50 |
| cerifera(carnauba) wax | | | | |
| cera alba | 0.50 | 0.50 | 0.50 | 0.40 |
| C16-40 alkyl stearate | | 1.50 | 1.50 | 1.50 |
| active ingredient | 0.5 | 3.0 | 1.0 | 5.0 |
| Uvinul ® A Plus ™ | 2.00 | 1.50 | 0.75 | 9.00 |
| UvaSorb ® k2A ethylhexyl | | 2.00 | | 4.00 |
| bis-isopentyl benzoxazolylphenyl melamine | | | | |
| ethylhexyl | | 3.00 | | |
| methoxycinnamate | | | | |
| Uvinul ® MC 80 | | | | |
| bis-ethylhexyloxyphenol | | 1.50 | | 2.00 |
| methoxyphenyl triazine | | | | |
| butyl methoxydi | | | 2.00 | |
| benzoylmethane | | | | |
| dinatrium phenyl | 2.50 | | 0.50 | 2.00 |
| dibenzimidazole | | | | |
| tetrasulfonate | | | | |
| ethyhexyl triazone | 4.00 | | 3.00 | 4.00 |
| Uvinul ® T 150 | | | | |
| octocrylene | | 4.00 | | |
| diethylhexyl butamido | 1.00 | | | 2.00 |
| triazone | | | | |
| phenylbenzimidazol | 0.50 | | 3.00 | |
| sulfonic acid | | | | |
| methylene bis- | 2.00 | | 0.50 | 1.50 |
| benzotriazolyl | | | | |
| tetramethylbutylphenol | | | | |
| ethylhexyl salicylate | | | 3.00 | |
| drometrizol trisiloxane | | | 0.50 | |
| terephthaliden dicamphor | | 1.50 | | 1.00 |
| sulfonic acid | | | | |
| diethylhexyl 2,6- | | | 7.00 | |
| naphthalate | | | | |
| titanium dioxide- | 1.00 | | 3.00 | |
| microfine | | | | |
| zinc oxide-microfine | | | | 0.25 |
| vitamin E acetate | 0.50 | 1.00 | | |
| ascorbyl palmitate | 0.05 | | 0.05 | |
| Buxux chinensis (jojoba) | 2.00 | 1.00 | | 1.00 |
| oil | | | | |
| perfume oil, BHT | 0.10 | 0.25 | | 0.35 |
| Ricinus communis | ad 100 | ad 100 | ad 100 | ad 100 |
| (castor) oil | | | | |

In a further preferred embodiment, a cosmetic composition comprises a PIT emulsion, which may contain, for example, the following ingredients in % in accordance with the International Nomenclature of Cosmetic Ingredients, INCI:

| Ingredients (in %) | Expl. 1 | Expl. 2 | Expl. 3 | Expl. 4 | Expl. 5 | Expl. 6 | Expl. 7 | Expl. 8 |
|---|---|---|---|---|---|---|---|---|
| glyceryl monostearate | 0.50 | 2.00 | 3.00 | 5.00 | | 0.50 | 4.00 | |
| SE | | | | | | | | |
| glyceryl isostearate | | | | | 3.50 | 4.00 | 2.00 | |
| isoceteth-20 | | 0.50 | | | 2.00 | | | |
| ceteareth-12 | | 5.00 | | 1.00 | | | 3.50 | 5.00 |
| ceteareth-20 | | 5.00 | | 1.00 | | | | 3.50 |
| PEG-100 stearate | | | | 2.80 | | 2.30 | 3.30 | |
| cetyl alcohol | 5.20 | | 1.20 | 1.00 | 1.30 | | 0.50 | 0.30 |
| cetyl palmitate | 2.50 | 1.20 | | 1.50 | | 0.50 | | 1.50 |
| cetyl dimethicone | | | | 0.50 | | 1.00 | | |
| copolyol | | | | | | | | |
| polyglyceryl-2- | | | | 0.75 | 0.30 | | | |
| dioleate | | | | | | | | |
| active ingredient | 0.1 | 5.0 | 0.01 | 0.5 | 3.0 | 0.25 | 10.0 | 3.0 |
| Uvinul ® A Plus ™ | 2.00 | 1.50 | 0.75 | 1.00 | 2.10 | 4.50 | 5.00 | 2.10 |
| UvaSorb ® k2A | | | 4.00 | | | | 1.50 | |
| ethylhexyl bis- | | | | | | | | |
| isopentylbenzoxazolyl | | | | | | | | |
| phenyl melamine | | | | | | | | |
| ethylhexyl | | | | | 5.00 | 6.00 | 8.00 | 5.00 |
| methoxycinnamate | | | | | | | | |
| Uvinul ® MC 80 | | | | | | | | |
| bis- | | 1.50 | | 2.00 | 2.50 | | 2.50 | 2.50 |
| ethylhexyloxyphenol | | | | | | | | |
| methoxyphenyl | | | | | | | | |
| triazine | | | | | | | | |
| butyl | | | 2.00 | | 2.00 | 1.50 | | 2.00 |
| methoxydibenzoylmethane | | | | | | | | |
| dinatrium phenyl | 2.50 | | 0.50 | 2.00 | | 0.30 | | |
| dibenzimidazole | | | | | | | | |
| tetrasulfonate | | | | | | | | |
| ethyhexyl triazone | 4.00 | | 3.00 | 4.00 | | 2.00 | | |
| Uvinul ® T 150 | | | | | | | | |
| octocrylene | | 4.00 | | | | | 7.50 | |
| diethylhexyl butamido | 1.00 | | | 2.00 | 1.00 | | 1.00 | 1.00 |
| triazone | | | | | | | | |
| phenylbenzimidazol | 0.50 | | 3.00 | | | | | |
| sulfonic acid | | | | | | | | |
| methylene bis- | 2.00 | | 0.50 | 1.50 | 2.50 | | | 2.50 |
| benzotriazolyl | | | | | | | | |
| tetramethylbutylphenol | | | | | | | | |
| ethylhexyl salicylate | | | 3.00 | | | | 5.00 | |
| drometrizol trisiloxane | | | 0.50 | | | 1.00 | | |
| terephthalylidene | | 1.50 | | 1.00 | 1.00 | | 0.50 | 1.00 |
| dicamphor sulfonic | | | | | | | | |
| acid | | | | | | | | |
| diethylhexyl 2,6- | | | 7.00 | | 10.00 | 7.50 | | 8.00 |
| naphthalate | | | | | | | | |
| titanium dioxide- | 1.00 | | 3.00 | | 3.50 | | 1.50 | 3.50 |
| microfine | | | | | | | | |
| zinc oxide-microfine | | | | 0.25 | | 2.00 | | |
| C12-15 alkyl | 3.50 | | | 6.35 | | | | 0.10 |
| benzoate | | | | | | | | |
| cocoglyceride | | 3.00 | | 3.00 | | | | 1.00 |
| dicapryl ether | 4.50 | | | | | | | |
| dicaprylyl carbonate | | 4.30 | | 3.00 | | | | 7.00 |
| dibutyl adipate | | | | 0.50 | | | | 0.30 |
| phenyl trimethicone | 2.00 | | | 3.50 | | 2.00 | | |
| cyclomethicone | | 3.00 | | | | | | |
| C1-5 alkyl | | 0.50 | | | 2.00 | | | |
| galactomannan | | | | | | | | |
| hydrogenated coco- | | | | | 3.00 | 4.00 | | |
| glycerides | | | | | | | | |
| behenoxy | | | | | | 1.50 | 2.00 | |
| dimethicone | | | | | | | | |
| VP/hexadecene | | | | 1.00 | 1.20 | | | |
| copolymer | | | | | | | | |
| glycerol | 4.00 | 6.00 | 5.00 | | 8.00 | 10.00 | | |
| vitamin E acetate | 0.20 | 0.30 | 0.40 | | 0.30 | | | |
| butyrospermum parkii | | 2.00 | | 3.60 | | 2.00 | | |
| (shea butter) | | | | | | | | |
| iodopropyl | 0.12 | | | | 0.20 | | | |
| butylcarbamate | | | | | | | | |
| biosaccaride gum-1 | | | | 0.10 | | | | |
| DMDM hydantoin | 0.10 | | | | 0.12 | | 0.13 | |
| methylparaben | | 0.50 | 0.30 | | 0.35 | | | |
| phenoxyethanol | 0.50 | 0.40 | | 1.00 | | | | |
| ethylhexylglycerol | | 0.30 | | | 1.00 | | 0.35 | |
| ethanol | 2.00 | | 2.00 | | | 5.00 | | |
| perfume oil | 0.20 | | 0.20 | | 0.24 | 0.16 | 0.10 | 0.10 |
| aqua dem. | ad 100 | ad 100 | ad 100 | ad 100 | ad 100 | ad 100 | ad 100 | ad 100 |

In a further preferred embodiment, a cosmetic composition comprises a gel creme, which may contain, for example, the following ingredients in % in accordance with the International Nomenclature of Cosmetic Ingredients, INCI:

| Ingredients (in %) | Example 1 | Example 2 | Example 3 | Example 4 |
|---|---|---|---|---|
| acrylates/C10-30 | 0.40 | 0.35 | 0.40 | 0.35 |
| alkylacrylate | | | | |
| crosspolymer | | | | |
| carbomer | 0.20 | 0.22 | 0.20 | 0.22 |
| Luvigel ® EM | 1.50 | 2.50 | 2.80 | 3.50 |
| xanthan gum | 0.10 | 0.13 | 0.10 | 0.13 |
| cetearyl alcohol | 3.00 | 2.50 | 3.00 | 2.50 |
| C12-15 alkylbenzoate | 4.00 | 4.50 | 4.00 | 4.50 |
| caprylic/capric | 3.00 | 3.50 | 3.00 | 3.50 |
| triglyceride | | | | |
| titanium | 1.00 | | 1.50 | |
| dioxide-microfine | | | | |
| zinc oxide-microfine | | 2.00 | | 0.25 |
| active ingredient | 0.5 | 10.0 | 3.0 | 5.0 |
| dihydroxyacetone | | | 3.00 | 5.00 |
| cyclomethicone | 5.00 | 5.50 | 5.00 | 5.50 |
| dimethicone | 1.00 | 0.60 | 1.00 | 0.60 |
| glycerol | 1.00 | 1.20 | 1.00 | 1.20 |
| sodium hydroxide | q.s. | q.s. | q.s. | q.s. |
| preservative | 0.30 | 0.23 | 0.30 | 0.23 |
| perfume oil | 0.20 | | 0.20 | |
| aqua dem. | ad 100 | ad 100 | ad 100 | ad 100 |

pH adjusted to 6.0

In a further preferred embodiment, a cosmetic composition comprises a hydrodispersion after sun, which may contain, for example, the following ingredients in % in accordance with the International Nomenclature of Cosmetic Ingredients, INCI:

| Ingredients (in %) | Example 1 | Example 2 | Example 3 | Example 4 | Example 5 |
|---|---|---|---|---|---|
| ceteaereth-20 | 1.00 | | | 0.50 | |
| cetyl alcohol | | | 1.00 | | |
| Luvigel ® EM | | 2.00 | | 2.50 | 2.00 |
| acrylates/C10-30 alkyl | 0.50 | 0.30 | 0.40 | 0.10 | 0.50 |
| acrylate crosspolymer | | | | | |
| xanthan gum | | 0.30 | 0.15 | | |
| active ingredient | 0.1 | 5.0 | 0.5 | 3.0 | 1.0 |
| C12-15 alkyl benzoate | 2.00 | 2.50 | | | |
| dicapryl ether | | 4.00 | | | |
| butylenglycol | 4.00 | | 2.00 | 6.00 | |
| dicaprylate/dicaprate | | | | | |
| dicapryl carbonate | | 2.00 | 6.00 | | |
| dimethicone | | 0.50 | 1.00 | | |
| phenyl trimethicone | 2.00 | | 0.50 | | |
| tricontanyl pvp | 0.50 | | 1.00 | | |
| ethylhexylglycerol | | | 1.00 | | 0.80 |
| glycerol | 3.00 | 7.50 | | 7.50 | 8.50 |
| glycine soja (soybean) oil | | | 1.50 | | 1.00 |
| vitamin E acetate | 0.50 | | 0.25 | | 1.00 |
| glucosylrutin | 0.60 | | | 0.25 | |
| trisodium EDTA | | 0.01 | 0.05 | | 0.10 |
| ethanol | 15.00 | 10.00 | 8.00 | 12.00 | 9.00 |
| perfume oil | 0.20 | | 0.05 | 0.40 | |
| aqua dem. | ad 100 | ad 100 | ad 100 | ad 100 | ad 100 |

In a further preferred embodiment, a cosmetic composition comprises a W/O emulsion, which may contain, for example, the following ingredients in % in accordance with the International Nomenclature of Cosmetic Ingredients, INCI:

| Ingredients (in %) | Example 1 | Example 2 | Example 3 | Example 4 | Example 5 |
|---|---|---|---|---|---|
| cetyl dimethicone | | 2.50 | | 4.00 | |
| polyglyceryl-2 | 5.00 | | | | 4.50 |
| dipolyhydroxystearate | | | | | |
| PEG-30 | | | 5.00 | | |
| dipolyhydroxystearate | | | | | |
| active ingredient | 5.0 | 10.0 | 0.1 | 0.5 | 1.0 |
| Uvinul ® A Plus ™ | 2.00 | 1.50 | 0.75 | 1.00 | 2.10 |
| titanium dioxid e- | 1.00 | | 3.00 | | 3.50 |
| microfine | | | | | |
| zinc oxide-microfine | | 0.90 | | 0.25 | |
| minera oil | | 12.00 | 10.00 | | 8.00 |
| C12-15 alkyl benzoate | | | | 9.00 | |
| dicaprylyl ether | 10.00 | | | | 7.00 |
| butylenglycol | | | 2.00 | 8.00 | 4.00 |
| dicaprylate/dicaprate | | | | | |
| dicaprylyl carbonate | 5.00 | | 6.00 | | |
| dimethicone | | 4.00 | 1.00 | 5.00 | |
| cyclomethicone | 2.00 | 25.00 | | | 2.00 |
| butyrospermum parkii | | | 3.00 | | |
| (shea butter) | | | | | |
| petrolatum | | 4.50 | | | |
| VP/hexadecene | 0.50 | | | 0.50 | 1.00 |
| copolymer | | | | | |
| ethylhexylglycerol | | 0.30 | 1.00 | | 0.50 |
| glycerol | 3.00 | 7.50 | | 7.50 | 8.50 |
| glycine soja (soybean) oil | | 1.00 | 1.50 | | 1.00 |
| magnesium sulfate | 1.00 | 0.50 | | 0.50 | |
| magnesium chloride | | | 1.00 | | 0.70 |
| vitamin E acetate | 0.50 | | 0.25 | | 1.00 |
| ascorbyl palmitate | 0.50 | | | 2.00 | |
| biosaccaride gum-1 | | | | 3.50 | 7.00 |
| DMDM hydantoin | | 0.60 | 0.40 | 0.20 | |
| methylparaben | 0.50 | | 0.25 | 0.15 | |
| phenoxyethanol | 0.50 | 0.40 | | 1.00 | |
| trisodium EDTA | 0.12 | 0.05 | | 0.30 | |
| ethanol | 3.00 | | 1.50 | | 5.00 |
| perfume oil | 0.20 | | 0.40 | 0.35 | |
| aqua dem. | ad 100 | ad 100 | ad 100 | ad 100 | ad 100 |

In a further preferred embodiment, a cosmetic composition comprises a pickering emulsion, which may contain, for example, the following ingredients in % in accordance with the International Nomenclature of Cosmetic Ingredients, INCI:

| Ingredients (in %) | Example 1 | Example 2 | Example 3 | Example 4 | Example 5 |
|---|---|---|---|---|---|
| mineral oil | | | 16.00 | 16.00 | |
| octyldodecanol | 9.00 | 9.00 | 5.00 | | |
| caprylic/capric | 9.00 | 9.00 | 6.00 | | |
| triglyceride | | | | | |
| C12-15 alkyl benzoate | | | | 5.00 | 8.00 |
| butylene glycol | | | | | 8.00 |
| dicaprylate/dicaprate | | | | | |
| dicaprylyl ether | 9.00 | | | 4.00 | |
| dicaprylyl carbonate | | 9.00 | | | |
| hydroxyoctacosanyl | 2.00 | 2.00 | 2.20 | 2.50 | 1.50 |
| hydroxystearate | | | | | |
| disteardimonium | 1.00 | 0.75 | | 0.50 | 0.25 |
| hectorite | | | | | |
| cera microcristallina + paraffinum | | 0.35 | | | 5.00 |
| liquidum | | | | | |
| hydroxypropyl | | | 0.10 | | 0.05 |
| methylcellulose | | | | | |
| dimethicone | | | | | 3.00 |
| active ingredient | 1.0 | 0.5 | 0.1 | 3.0 | 5.0 |
| titanium dioxide + alumina + simethicone + aqua | | 3.00 | | | |
| titanium dioxide + trimethoxycaprylylsilane | | 2.00 | 4.00 | 2.00 | 4.00 |
| silica dimethyl silylate | 2.50 | | | 6.00 | 2.50 |
| boron nitride | | | 1.00 | | |
| starch/sodium | 2.00 | | | | |
| metaphosphate polymer | | | | | |
| tapioca starch | | 0.50 | | | |
| sodium chloride | 5.00 | 7.00 | 8.50 | 3.00 | 4.50 |
| glycerol | | | | 1.00 | |
| trisodium EDTA | 1.00 | 1.00 | 1.00 | 1.00 | 1.00 |
| vitamin E acetate | 5.00 | 10.00 | 3.00 | 6.00 | 10.00 |
| ascorbyl palmitate | 1.00 | 1.00 | | 1.00 | |
| methylparaben | | 0.60 | | | 0.20 |
| propylparaben | | | | | 0.20 |
| phenoxyethanol | | | 0.20 | | |
| hexamidine diisethionate | | | 0.40 | 0.50 | 0.40 |
| diazolidinyl urea | | | | | 0.08 |
| ethanol | | | 0.23 | 0.20 | |
| perfume oil | 5.00 | | 3.00 | 4.00 | |
| aqua dem. | 0.20 | | 0.30 | 0.10 | |

In a further preferred embodiment, a cosmetic composition comprises a stick, which may contain, for example, the following ingredients in % in accordance with the International Nomenclature of Cosmetic Ingredients, INCI:

| Ingredients (in %) | Example 1 | Example 2 | Example 3 | Example 4 |
|---|---|---|---|---|
| caprylic/capric | 12.00 | 10.00 | 6.00 | |
| triglyceride | | | | |
| octyldodecanol | 7.00 | 14.00 | 8.00 | 3.00 |
| butylene glycol | | | | 12.00 |
| dicaprylate/dicaprate | | | | |
| pentaerythrityl | 10.00 | 6.00 | 8.00 | 7.00 |
| tetraisostearate | | | | |
| polyglyceryl-3 | 2.50 | | | |
| diisostearate | | | | |
| bis-diglyceryl | 9.00 | 8.00 | 10.00 | 8.00 |
| polyacyladipate-2 | | | | |
| cetearyl alcohol | 8.00 | 11.00 | 9.00 | 7.00 |
| myristyl myristate | 3.50 | 3.00 | 4.00 | 3.00 |
| beeswax | 5.00 | 5.00 | 6.00 | 6.00 |
| copernicia | 1.50 | 2.00 | 2.00 | 1.50 |
| cerifera(carnauba) wax | | | | |
| cera alba | 0.50 | 0.50 | 0.50 | 0.40 |
| C16-40 alkyl stearate | | 1.50 | 1.50 | 1.50 |
| active ingredient | 10.0 | 1.0 | 3.0 | 0.1 |
| Uvinul ® A Plus ™ | 2.00 | 1.50 | 0.75 | 9.00 |
| titanium dioxide- | 1.00 | | 3.00 | |
| microfine | | | | |
| zinc oxide-microfine | | 1.00 | | 0.25 |
| vitamin E acetate | 0.50 | 1.00 | | |
| ascorbyl palmitate | 0.05 | | 0.05 | |
| Buxux chinensis (jojoba) | 2.00 | 1.00 | | 1.00 |
| oil | | | | |
| perfume oil, BHT | 0.10 | 0.25 | | 0.35 |
| Ricinus communis | ad 100 | ad 100 | ad 100 | ad 100 |
| (castor) oil | | | | |

In a further preferred embodiment, a cosmetic composition comprises an oil gel, which may contain, for example, the following ingredients in % in accordance with the International Nomenclature of Cosmetic Ingredients, INCI:

| Ingredients (in %) | Example 1 | Example 2 | Example 3 | Example 4 |
|---|---|---|---|---|
| caprylic/capric | 12.00 | 10.00 | 6.00 | |
| triglyceride | | | | |
| octyldodecanol | 7.00 | 14.00 | 8.00 | 3.00 |
| butylene glycol | | | | 12.00 |
| dicaprylate/dicaprate | | | | |
| pentaerythrityl | 10.00 | 6.00 | 8.00 | 7.00 |
| tetraisostearate | | | | |
| polyglyceryl-3 | 2.50 | | | |
| diisostearate | | | | |
| bis-diglyceryl | 9.00 | 8.00 | 10.00 | 8.00 |
| polyacyladipate-2 | | | | |
| myristyl myristate | 3.50 | 3.00 | 4.00 | 3.00 |
| quaternium-18 bentonite | 5.00 | 5.00 | 6.00 | 6.00 |
| propylene carbonate | 15.00 | 20.00 | 18.00 | 19.50 |
| active ingredient | 1.0 | 0.5 | 3.0 | 5.0 |
| vitamin E acetate | 0.50 | 1.00 | | |
| ascorbyl palmitate | 0.05 | | 0.05 | |
| Buxus chinensis (jojoba) | 2.00 | 1.00 | | 1.00 |
| oil | | | | |
| perfume oil, BHT | 0.10 | 0.25 | | 0.35 |
| Ricinus communis | ad 100 | ad 100 | ad 100 | ad 100 |
| (castor) oil | | | | |

Phases A and B are heated separately to approx. 80° C. Subsequently, phase C is stirred into phases A and B and homogenized. Finally, the mixture is cooled down to 40° C. and phases D and E are added.

The present invention also relates to the use of a microorganism according to the invention or of a derivative, mutant or inactive form thereof as described herein above for the preparation of a pharmaceutical composition for preventing, suppressing or treating foot malodor, hyperhydrosis of the feet or athlete's foot.

In another aspect the present invention relates to the use of a microorganism according to the invention or of a derivative, mutant or inactive form thereof as described herein above in the context of textiles or textile substrates. Preferably, the present invention relates to the use of a microorganism according to the invention or of a derivative, mutant or inactive form thereof as described herein above for the conditioning or impregnation of textiles or textile substrates. More preferably, the microorganism according to the invention or a derivative, mutant or inactive form thereof as described herein above may be applied into or onto textiles or textile substrates according to any suitable methods known to the person skilled in the art or as exemplified herein below. Therefore the present invention also relates to any of the uses, compositions or methods as described herein above in the ambit of textiles or textile substrates.

Accordingly, the present invention relates to a method for the production of textiles and textile substrates for preventing the generation of foot malodor by skin microorganisms comprising the steps of formulating a microorganism according to the invention or a mutant, derivative or inactive form of this microorganism as described above with textiles and textile substrates. Preferably, said textiles and textile substrates may comprise a cosmetically or pharmaceutical acceptable carrier or excipient as described herein above or comprise one or more of the cosmetic or pharmaceutical compositions as described herein above.

The term “textile and textile substrates for preventing the generation of foot malodor by skin microorganisms”, as used in accordance with the present invention, relates to (a) textile composition(s) which comprise(s) at least one microorganism of the present invention or mutant, derivative or inactive form of said microorganism as described above. It is envisaged that the textile compositions of the present invention comprise the aforementioned ingredients in any combination. It may, optionally, comprise at least one further ingredient suitable for preventing the generation of foot malodor by skin microorganisms (see also Ullmann, Vol. A 26 S. 227 if, 1995, which is incorporated herein by reference).

According to the present invention, textiles and textile substrates are textile fibres, semi-finished and finished textiles and finished products produced therefrom also comprising—apart from textiles for the clothing industry—for example, carpets and other home fabrics and textile formations serving technical purposes. These formations also include unshaped formations such as flocks, linear formations such threads, fibres, yarns, linens, cords, ropes, ply yarns and solid formations such as, for example, felts, woven fabrics, hosiery, knitted fabrics, bonded fibre fabrics and wadding. The textiles can be made, for example, of materials of natural origin, e.g., cotton wool, wool or flax, or synthetic, e.g., polyamide, polyester, modified polyester, polyester blended fabrics, polyamide blended fabrics, polyacrylonitrile, triacetate, acetate, polycarbonate, polypropylene, polyvinyl chloride, polyester microfibres or glass fibre fabrics.

In an embodiment of the present invention, the method for the production of textiles and textile substrates for preventing the generation of foot malodor by skin microorganisms according to the invention may be carried out with any machine or apparatus for the finishing of textiles known to the skilled person, for example standard machines such as foulards. Preferably said foulards are foulard machines with, e.g., vertical infeed, which contain, for example, as essential element two rolls pressed together through which the textile is guided. Above the rolls, an aqueous formulation may be filled in which moistens the textile. Typcically, the pressure quetches the textile and ensures a constant application. In another preferred embodiment, in the foulard machines the textile is, for instance, guided first through an immersion bath and subsequently upwards through two rolls pressed together, e.g. in foulards with vertical textile infeed from below. Machines or apparatuses for the finishing of textiles, especially foulard machines, are described, for example, in Hans-Karl Rouette, “Handbuch der Textilveredlung”, Deutscher Fachverlag 2003, p. 618 to 620 which herein incorporated by reference.

In a further embodiment of the present invention, the method for the production of textiles and textile substrates for preventing the generation of foot malodor by skin microorganisms according to the invention can be carried out according to any suitable exhaustion method known to the person skilled in the art, such as, for example, spraying, slop padding, kiss-roll or printing. Prefrably, the method for the production of textiles and textile substrates for preventing the generation of foot malodor by skin microorganisms according to the invention is carried out according to an exhaustion method with a liquor absorption, for example, in the range from 1 to 50%, preferably from 20 to 40%.

In a further embodiment of the present invention, the textile can subsequently be treated thermally by any suitable means known to the person skilled in the art, for example by drying at temperatures in the range of 30 to 100° C. or by thermal fixing at temperatures in the range of at least 100, preferably at least 101° C. up to 150° C., preferably up to 135° C. In a preferred embodiment, the treatment may be thermal over a period of 10 seconds up to 30 minutes, preferably 30 seconds up to 10 minutes. In further preferred embodiment of the present invention, two thermal treatment steps are carried out at different temperatures, for example, in the first step, drying takes place at temperatures in the range of, e.g., 30 to 100° C. over a period of, e.g., 10 seconds to 20 minutes, and then fixing takes place at temperatures in the range of, e.g., 101 to 135° C. over a period of, e.g., 30 seconds to 3 minutes.

In a preferred embodiment, the further ingredient comprised in the textile and textile substrates according to the present invention which is suitable for preventing the generation of foot malodor by skin microorganisms may be a cyclodextrin as described in DE 40 35 378 or DE 10101294.2 or amylose-containing substances as described in EP-A1-1522626.

Typically, cyclodextrins are cyclic oligosaccharides which are formed by the enzymatic degradation of starch. Preferably, the cyclodextrins to be used as ingredients in the textiles or textile substrates according to the invention are [alpha]-, [beta]- or [gamma]-cyclodextrins which consist, for instance, of six, seven or eight, respectively, [alpha]-1,4 linked glucose units. A characteristic property of the cyclodextrin molecules is their ring structure with largely constant dimensions. Typically, the internal diameter of the rings is about 570 pm for [alpha]-cyclodextrin, about 780 pm for [beta]-cyclodextrin and about 950 pm for [gamma]-cyclodextrin. Due to their structure, cyclodextrins are in the position to be able to incorporate guest molecules. In a preferred embodiment these guest molecules comprise volatile fragrances as known to the person skilled in the art.

In a further preferred embodiment the present invention provides the use of amylose-containing substances for modifying the odour properties of textiles or textile substrates according to the invention. Preferably, the amylose content is at least 30% by weight, based on the total weight of the substance. The invention also provides a method of modifying the odour properties of textiles which is characterized in that the textile is finished with amylose or an amylose-containing substance, preferably with an amylose content of at least 30% by weight. The term “amylose or amylose-containing substance” means any amylose-containing starches, e.g. native starches, modified starches and starch derivatives, whose amylose content is preferably at least 30% by weight. The starch may be native, e.g. maize starch, wheat starch, potato starch, sorghum starch, rice starch or maranta starch, be obtained by partial digestion of native starch or be chemically modified. Also suitable is pure amylose as it is, e.g. enzymatically obtained amylose, e.g. amylose obtained from sucrose. Also suitable are mixtures of amylose and starch, preferably if the total content of amylose is at least 30% by weight, based on the total weight of the mixture. All data in % by weight which refers to amylose or amylose-containing substances, for mixtures of amylose and starch are always based on the total weight of amylose+starch, unless expressly stated otherwise.

Of particular suitability according to the invention are amylose-containing substances, in particular amylose and amylose-containing starches, and amylose/starch mixtures whose amylose content is at least 40% by weight and in particular at least 45% by weight, based on the total weight of the substance. As a rule, the amylose content will not exceed 90% by weight and in particular 80% by weight. Such substances are known to the person skilled in the art and commercially available.

To achieve the odour-modifying effect, the textile according to the invention may be finished with the amylose-containing substance generally in any suitable amount, known to the person skilled in the art, preferably of at least 0.5% by weight, more preferably at least 1% by weight and in particular at least 2% by weight, in each case based on the weight of the textile. As a rule, the amylose-containing substance may be used in an amount of not more than 25% by weight, often not more than 20% by weight and in particular not more than 15% by weight, based on the weight of the textile so as not to adversely affect the tactile properties of the textile.

In a further preferred embodiment of the invention, to improve the odour properties, the textile material according to the invention may be finished with the amylose-containing substance as it is. However, it is also possible to use the amylose-containing substance together with a fragrance in order to achieve a long-lasting pleasant odour, or scent of the textile. Preferably, the procedure involves treating the textile according to the invention with the amylose-containing substance or to treat the textile with the microorganism according to the present invention and the amylose-containing substance at the same time. The textile finished in this way will then be treated with the fragrance. As a result, the amylose-containing substance is charged with the fragrance.

In a further preferred embodiment the textile or textile substrate according to the invention which is formulated with a microorganism according to the invention or a mutant, derivative or inactive form of this microorganism as described above may be finished with a frangrance.

Preferably, the fragrance as used according to any of the above embodiments may be used in an amount which suffices for the desired scent effect, as known to the person skilled in the art. The upper limit is determined by the maximum absorption capacity of the amylose units of the amylose-containing substance used and will generally not exceed 20% by weight and often 10% by weight, based on the amylose content of the substance. If desired, the fragrance is generally used in an amount of from 0.1 to 10% by weight and in particular 0.5 to 5% by weight.

Suitable fragrances are in principle all volatile organic compounds and mixtures of organic compounds which are known as fragrances. A review of fragrances is given in Ullmann's Encyclopedia of Industrial Chemistry, 5th ed. on CD Rom, Flavours and Fragrances, chapter 2, in particular chapters 2.1 to 2.4. Of particular suitability according to the invention are fragrances of aliphatic and cycloaliphatic nature. These include: aliphatic C4-C12-alcohols, e.g. 3-octanol, cis-3-hexen-1-ol, trans-3-hexen-1-ol, 1-octen-3-ol, 2,6-dimethylheptan-2-ol, 1-octen-3-ol, 9-decen-1-ol, 10-undecen-1-ol, 2-trans-6-cis-nonadien-1-ol, aliphatic C6-C13-aldehydes, e.g. hexanal, octanal, nonanal, decanal, undecanal, 2-methyldecanal, 2-methylundecanal, dodecanal and tridecanal, cis-4-heptenal and 10-undecenal, esters of aliphatic C1-C6-carboxylic acids with aliphatic, optionally monounsaturated C1-C8-alcohols such as ethyl formate, cis-3-hexenyl formate, ethyl acetate, butyl acetate, isoamyl acetate, hexyl acetates, 3,5,5-trimethylhexyl acetate, trans-2-hexenyl acetate, cis-3-hexenyl acetate, ethyl propionate, ethyl butyrates, butyl butyrate, isoamyl butyrate, hexyl butyrate, cis-3-hexenyl isobutyrate, ethyl isovalerate, ethyl 2-methylbutyrate, ethyl hexanoate, 2-propenyl hexanoate, ethyl heptanoate, 2-propenyl heptanoate and ethyl octanoate, acyclic terpene hydrocarbons and hydrocarbon alcohols, such as nerol, geraniol, tetrahydrogeraniol, linalool, tetrahydrolinalool, citronellol, lavandulol, myrcenol, farnesol, nerolidol, the formates, acetates, propionates, butyrates, valerates and isobutyrates of these alcohols, the aldehydes corresponding to the abovementioned alcohols, such as citral, citronellal, hydroxydihydrocitronellal, methoxydihydrocitronellal and the dimethyl- and diethylacetals of these aldehydes, such as diethylcitral, methoxydihydrocitronellal-dimethylacetal, also cyclic terpene hydrocarbons, hydrocarbon alcohols and aldehydes. These may also include scents of natural provenance, such as rose oil, lemon oil, lavender oil and oil of cloves scent.

Thus, the present inventin also relates to textiles or textile substrates comprising a microorganism according to the invention or of a derivative, mutant or inactive form thereof as described herein above. “Comprising” may, e.g., mean associated with or incorporating the microorganism according to the invention or a derivative, mutant or inactive form thereof as described herein above, in particular, in a form as it results from one of the above-described methods.

In another aspect the present invention relates to a method for the production of a composition comprising the step of formulating a microorganism of the invention or a derivative or mutant thereof or an inactive form as described herein above with a cosmetically and/or pharmaceutically carrier or excipient.

The present invention furthermore relates to a method of preventing, suppressing or treating foot malodor, comprising the step of administering to a patient in need thereof a prophylactically or therapeutically effective amount of a composition according to the invention.

It is to be understood that this invention is not limited to the particular methodology, protocols, bacteria, vectors, and reagents etc. described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

Preferably, the terms used herein are defined as described in “A multilingual glossary of biotechnological terms: (IUPAC Recommendations)”, Leuenberger, H. G. W, Nagel, B. and Kölbl. H. eds. (1995), Helvetica Chimica Acta, CH-4010 Basel, Switzerland). Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step.

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention.

It must be noted that as used herein and in the appended claims, the singular forms “a”, “an”, and “the”, include plural referents unless the context clearly indicates otherwise. Thus, for example, reference to “a reagent” includes one or more of such different reagents, and reference to “the method” includes reference to equivalent steps and methods known to those of ordinary skill in the art that could be modified or substituted for the methods described herein.

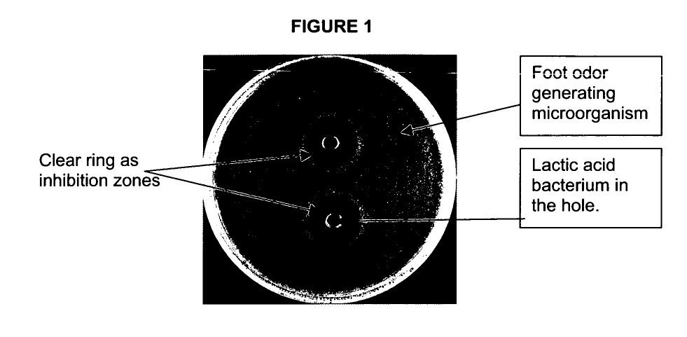

FIG. 1 shows the inhibition of foot odor generating microorganisms (Example 3) in an in vitro-hole plate assay. Shown are agar plates with the foot odor generating indicator strain Micrococcus spec. and lactic acid bacterium Lactobacillus brevis, LB-FG-0009 (DSM 17607). The formation of a black ring around the well indicates growth inhibition of the indicator strain Micrococcus spec.

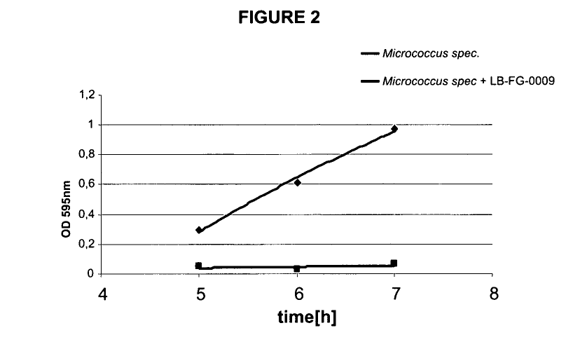

FIG. 2 shows the growth inhibition of foot odor generating microorganism Micrococcus spec. in an in-vitro-liquid assay (Example 4). Lactobacillus brevis, LB-FG-0009 (DSM 17607) was used. Shown is the degree of inhibition as quantified by counting the colony forming units of the indicator strain in comparison to the control without lactic acid bacteria

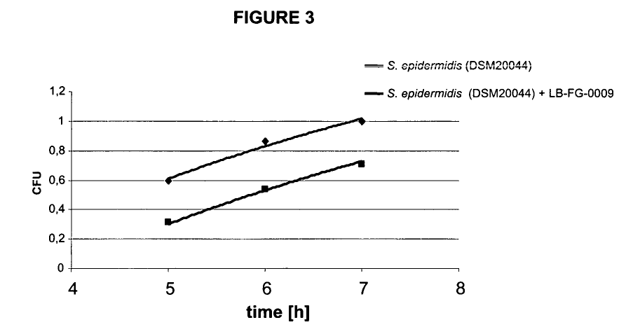

FIG. 3 shows the lack of growth inhibition of Staphylococcus epidermidis (DSM20044) in an in-vitro- liquid assay (Example 5). Lactobacillus brevis, LB-FG-0009 (DSM 17607) was used. Shown is the degree of inhibition as quantified by counting the colony forming units of the indicator strain in comparison to the control without lactic acid bacteria.

A better understanding of the present invention and of its advantages will be obtained from the following examples, which are offered for illustrative purposes only and are not intended to limit the scope of the present invention in any way.

### EXAMPLE 1

### Identification for Odor Generating Microorganisms/Odor Assay

The biosynthetic pathway involved in the generate of isovaleric acid by microorganism is known (Thierry et al., Appl. Env. Microbiol. 68(2) (2002), 608-615), but corresponding feet microorganisms that are able to generate this substance are unknown. The inventors have identified specific microorganisms that are involved in the generation of foot odor. To identify feet microorganisms that are involved in the generation of foot odor due to the synthesis of the odorous substance isovaleric acid, microorganisms were isolated from subjects with foot odor.

### Isolation of Feet Microorganisms

Feet microorganisms were isolated from the skin by rubbing a sterile Q-tip on a defined region (2 cm ×2 cm) on the bottom of the foot. The Q-tip was transferred to a sterile buffer solution (PBS, pH 7.0) and dilutions were plated on selective culture agar plates for either gram positive (e.g. BHl, Difco Inc.) or gram negative bacteria (e.g. MacConkey agar, Difco Inc.) or to a selective culture agar for yeasts and fungi (e.g. Plate Count Agar, Difco Inc.). Afterwards the microorganisms that have been transferred from skin to culture agar plates were cultivated at 30° C. and 37° C., aerobically and anaerobically for about 24 hours. Colony forming units were determined by morphological and biochemical methods for a qualitative analysis and by counting for quantification. The relative composition and total cell counts were determined.

### Identification of Odor Generating Microorganisms/Odor Assay

Individual microorganisms are responsible for the generation of typical foot odor due to the biosynthesis of isovaleric acid. These microorganisms are able to generate isovaleric acid due to metabolic activities. The following assay was performed to identify microorganisms which are able to synthesize isovaleric acid.

Isolated microorganisms were cultivated in 20 ml corresponding culture broths (for gram positives, gram negatives, yeast or fungi (Difco Inc.)) for 24 h at 30° C./37° C. aerobically or anaerobically. Cells were centrifuged (4000×g) and washed two times with 60 mM phosphate buffer, pH 8.0. for the odor generation assay cells were suspended in 60 mM phosphate buffer, ph 8.0, containing 5 mM L-leucine and 10 mM alpha-ketoglutarate and were incubated aerobically for 24 h at vigorous shaking (160 rpm rotary shaker). Afterwards cells were pelleted by centrifugation (4000×g, 5 min) and the supernatant was transferred to a glass bottle for gas chromatographic analysis. The generation of isovaleric acid was observed by GC analysis, performed on a Hewlett-Packard 5890 series II gas chromatograph with a capillary column (30 m by 0.5 mm by 0.53 μm [film thickness]; Agilent HP-FFAP). The temperature program was from 150° C. to 220° C. The temperature was initially 150° C. for 2 min; it was then increased at 15° C./min to a final temperature of 220° C., at which it was held for 3 min. The constant flow velocity was 30 cm/s, helium was used as the carrier gas, and injections were run in the splitless mode. Isovaleric acid was identified by gas chromatographic/mass spectrometric analysis and comparison of retention time to the pure standard substance.

The generation of typical cheesy odor was verified by sniffing of samples after reacidification with HCl. In detail after incubation an aliquot was reacidified by dropping 6 M HCl solution into the sample. Due to the acidification isovaleric acid evaporated and was recognized by the nose in a dose dependent manner. Those microorganisms which were able to generate isovaleric acid and the corresponding typical cheesy smell were classified as odor generating foot microorganisms.

### EXAMPLE 2

### Identification of Microorganisms Preventing the Generation of Isovaleric Acid by Microorganisms

Individual microorganisms are responsible for the generation of typical foot odor due to the biosynthesis of isovaleric acid. These microorganisms are able to generate isovaleric acid due to metabolic activities. A specific inhibition of the biosynthesis of isovaleric acid by microorganisms, in particular lactic acid bacteria have been identified, that are able to inhibit the biosynthesis of isovaleric acid by odor generating foot microorganisms. To test this effect the following assay has been performed.

### Cultivation and Preparation of Lactobacilli

Lactic acid bacteria were cultivated from a −80° C. freezing culture in 1 ml MRS broth in Eppendorf tubes. Tubes were closed and cultivated for 2 days at 37° C. 5 μl of this preculture was transferred to the main culture consisting of 0.5 ml MRS broth in Eppendorf tubes. The culture was incubated for two days. After cultivation cells were harvested by centrifugation (4000×g, 15 min). The cell pellet was washed two times with 60 mM phosphate buffer (pH 0.8). Cells were resuspended in 200 μl 60 mM phosphate buffer (pH 8.0).

### Cultivation and Preparation of the Indicator Strain

One exemplary indicator strain was Micrococcus spec. For cultivation 20 ml BHl broth in a shaking glass flask was inoculated with 15 μl of a 24 h preculture (20 ml). The indicator strain was cultivated for 24 h at 37° C. with vigorous shaking (160 rpm on a reciprocal shaker). Cells were harvested by centrifugation (4000×g, 5 min) and washed twice in PBS buffer (pH 8.0). For the following odor prevention assay cells were resuspended in 60 mM phosphate buffer, pH 8.0.

### Odor Prevention Assay

For the odor prevention assay 15 μl of the prepared indicator strain was aerobically incubated in the presence of 100 μl Lactobacillus culture, 60 mM phosphate buffer (pH 8.0) 5 mM L-leucine and 10 mM alpha-ketoglutarate for 24 h at 30° C. at vigorous shaking. Afterwards cells were pelleted by centrifugation (4000×g, 5 min) and the supernatant was transferred to a glass bottle for gas chromatographic analysis. Corresponding control samples were incubated without lactobacilli. The prevention of isovaleric acid generation was observed by GC analysis, performed on a Hewlett-Packard 5890 series II gas chromatograph with a capillary column (30 m by 0.5 mm by 0.53 μm [film thickness]; Agilent HP-FFAP). The temperature program was from 150° C. to 220° C. The temperature was initially 150° C. for 2 min; it was then increased at 15° C./min to a final temperature of 220° C., at which it was held for 3 min. The constant flow velocity was 30 cm/s, helium was used as the carrier gas, and injections were run in the splitless mode. Isovaleric acid was identified by gas chromatographic/mass spectrometric analysis and comparison of retention time to the pure standard substance.

The prevention of typical cheesy odor generation was verified by sniffing of sample after reacidification with HCl. In detail after incubation an aliquot was reacidified by dropping 6 M HCl into the sample. Due to the acidification isovaleric acid evaporated and was recognized by the nose in a dose dependent manner.

### EXAMPLE 3

### Inhibition of Odor Generating Feet Microorganisms

Individual microorganisms are responsible for the generation of typical foot odor due to the biosynthesis of isovaleric acid. The specific inhibition of these microorganisms by topically applied microorganisms without disturbing the complete microbial skin flora at the feet is an effective way to reduce the generation of foot odor while the skin microbial flora still exists to protect the skin. Specific lactic acid bacteria have been identified that are able to inhibit the growth of odor generating foot microorganisms on agar plates in an in-vitro-hole plate assay. To test this effect, precultured lactic acid bacteria were filled into pre-cutted holes and a growth inhibition of the indicator strain were observed. Inhibition was defined as the formation of a clear ring around the hole the lactic acid bacterium was pipetted in. For several strains it was observed that they inhibit the growth of foot microorganisms. Data for Lactobacillus brevis, LB-FG-0009 (DSM 17607) and indicator Micrococcus spec. are shown in FIG. 1.

### Cultivation and Preparation of Lactobacilli

Lactic acid bacteria were cultivated from a −80° C. freezing culture in 1 ml MRS broth in Eppendorf tubes. Tubes were closed and cultivated for 2 days at 37° C. 10 μl of this preculture was transferred to the main culture consisting of 7 ml MRS broth in Falcon tubes. The culture was incubated for one day. After cultivation cells were harvested by centrifugation (15 min, 4000×g). The cell pellet was washed twice with K/Na-buffer (each 1 ml). Cells were resuspended in 200 μl K/Na buffer.

### Cultivation and Preparation of the Indicator Strain

The indicator strain was Micrococcus spec. 20 ml BHl broth in a shaking glass flask was inoculated with 15 μl of a 24 h preculture. The indicator strain was cultivated for 24 h at 37° C. An aliquot was diluted to an optical density OD₅₉₅ₙₘ of 0.025-0.05 in BHl-broth and 800 μl were spread on indicator plates (BHl-Agar). The agar was stamped using a cork borer. The holes were filled with the pre-cultured lactic acid bacteria.

### Media and Buffer:

| BHI-Agar | Difco Agar 1.8%; 20 ml per plate | |
|---|---|---|
| BHI-Medium | Difco | |
| MRS-broth | Difco, 150 μl/well | |
| K/Na-buffer | according to Küster Thiel, ph 7.0, autoclaved | |
| | 0.066 M Na₂HPO₄ × 2H₂O | 61.2 ml |
| | 0.066 M KH₂PO₄ | 38.8 ml |

### EXAMPLE 4

### Growth Inhibition of Foot Odor Generating Microorganism in the In-Vitro—Liquid Assay

The selected Lactobacillus brevis (LB-FG-0009) is able to prevent the growth of foot malodour generating foot microorganisms in an in vitro liquid assay.

To test this effect, the pre cultured lactic acid bacterium has been co-incubated with the indictator strain in a liquid culture. The degree of inhibition was quantified by counting the colony forming units of the indicator strain in comparison to the control without lactic acid bacteria. Data are shown in FIG. 2.

### Cultivation and Preparation of Lactobacilli

Lactic acid bacteria were cultivated from a −80° C. freezing culture in 1 ml MRS broth in eppendorf tubes. Tubes were closed and cultivated for 2 days at 37° C. 10 μl of this pre culture was transferred to the main culture consisting of 7 ml MRS broth in falcon tubes. The culture was incubated for 2 days. After cultivation cells were harvested by centrifugation (15 min, 4000×g). The cell pellet was washed two times with K/Na-buffer (each 1 ml). Cells were resuspended in 200 μl K/Na buffer.

### Cultivation and Preparation of the Indicator Strain

The indicator strain was Micrococcus spec. 20 ml BHl broth in a shaking glass flask was inoculated with 15 μl of a freezing culture for a 24 h pre culture.

### Liquid Inhibition Assay

For the liquid assay 5 μl of the freshly prepared lactic acid bacteria (out of 200 μl) and 10 μl of the pre cultured indicator strain were inoculated for a co-cultivation in 10 ml of BHl broth. The culture was incubated for 7 h. Afterwards 100 μl of a 1:1000 dilution was spread on a BHl agar plate for quantification of the colony forming units. The plate was incubated for 24 h hours and the colony forming units were counted.

### Media and Buffer:

| BHI-Agar | Difco Agar 1.8%; 20 ml per plate | |
|---|---|---|
| BHI-Medium | Difco | |
| MRS-broth | Difco | |
| K/Na-buffer | according to Küster Thiel, pH 7.0, autoclaved | |
| | 0.066 M Na₂HPO₄ × 2H₂O | 61.2 ml |
| | 0.066 M KH₂PO₄ | 38.8 ml |

### EXAMPLE 5

### No Growth Inhibition of Staphylococcus Epidermidis (DSM20044) in the In-Vitro—Liquid Assay

The selected Lactobacillus brevis (LB-FG-0009) that is able to prevent the generation of foot malodour by foot microorganisms does not inhibit the important member of the commensal micro flora of the foot skin, Staphylococcus epidermidis (DSM20044), in an in vitro liquid assay.

To test this effect, the pre cultured lactic acid bacterium has been co-incubated with the indictator strain in a liquid culture. The degree of inhibition was quantified by counting the colony forming units of the indicator strain in comparison to the control without lactic acid bacteria. Data are shown in FIG. 3.

### Cultivation and Preparation of Lactobacilli

Lactic acid bacteria were cultivated from a −80° C. freezing culture in 1 ml MRS broth in eppendorf tubes. Tubes were closed and cultivated for 2 days at 37° C. 10 μl of this pre culture was transferred to the main culture consisting of 7 ml MRS broth in falcon tubes. The culture was incubated for 2 days. After cultivation cells were harvested by centrifugation (15 min, 4000×g). The cell pellet was washed two times with K/Na-buffer (each 1 ml). Cells were resuspended in 200 μl K/Na buffer.

### Cultivation and Preparation of the Indicator Strain

The indicator strain was Staphylococcus epidermidis (DSM20044) 20 ml BHl broth in a shaking glass flask was inoculated with 15 μl of a freezing culture for a 24 h pre culture.

### Liquid Inhibition Assay

For the liquid assay 5 μl of the freshly prepared lactic acid bacteria (out of 200 μl) and 10 μl of the pre cultured indicator strain S. epidermidis (DSM20044) were inoculated for a co-cultivation in 10 ml of BHl broth. The culture was incubated for 7 h. Afterwards 100 μl of a 1:1000 dilution was spread on a BHl agar plate for quantification of the colony forming units. The plate was incubated for 24 h hours and the colony forming units were counted.

### Media and Buffer:

| BHI-Agar | Difco Agar 1.8%; 20 ml per plate | |
|---|---|---|
| BHI-Medium | Difco | |
| MRS-broth | Difco | |
| K/Na-buffer | according to Küster Thiel, pH 7.0, autoclaved | |
| | 0.066 M Na₂HPO₄ × 2H₂O | 61.2 ml |
| | 0.066 M KH₂PO₄ | 38.8 ml |

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. A microorganism which is able to prevent the generation of foot malodor by skin microorganisms.

23. The microorganism of claim 22 which is able to prevent the generation of foot malodor by skin microorganisms in a sniffing assay.

24. The microorganism of claim 22 which is able to suppress the biosynthesis of isovaleric acid by skin microorganisms.

25. The microorganism of claim 24, which is able to suppress the biosynthesis of isovaleric acid by skin microorganisms in an assay comprising the following steps:
(i) mixing said microorganism with a microorganism which is capable of generating isovaleric acid and with a precursor of isovaleric acid;
(ii) incubating the mixture under conditions allowing the generation of isovaleric acid;
(iv) extracting short fatty acids from the supernatant of the mixture; and
(iv) detecting the generation of foot malodor by the occurrence of isovaleric acid.

26. The microorganism of claim 24, wherein isovaleric acid is generated by a bacterium of the genus Micrococcus or Propionibacterium.

27. The microorganism of claim 26, wherein the Propionibacterium is Propionibacterium freudenreichii.

28. The microorganism of claim 22, which is able to inhibit the growth of foot malodor generating skin microorganisms.

29. The microorganism of claim 28, which is able to inhibit the growth of foot malodor generating skin microorganisms in an inhibition assay.

30. The microorganism of claim 22, which belongs to a genus selected from the group consisting of Lactobacillus, Lactococcus and Leuconostoc.

31. The microorganism of claim 30, which is selected from the group consisting of Lactobacillus brevis, Lactobacillus plantarum, Lactobacillus curvatus, Lactobacillus delbrückii delbrückii, and Leuconostoc mesenteroides.

32. The microorganism of claim 31, which is selected from the group consisting of Lactobacillus brevis LB-FG-0001 (DSM 17599), Lactobacillus plantarum LB-FG-0002(DSM 17600), Lactobacillus curvatus LB-FG-0003 (DSM 17601), Leuconostoc mesenteroides LB-FG-0004 (DSM 17602), Lactobacillus plantarum LB-FG-0005 (DSM 17603), Lactobacillus delbrückii delbrückii LB-FG-0006 (DSM 17604), Lactobacillus delbrückii delbrückii LB-FG-0007 (DSM 17605), Lactobacillus plantarum LB-FG-0008 (DSM 17606) and Lactobacillus brevis, LB-FG-0009 (DSM 17607).

33. The microorganism of claim 31, which is a mutant of the selected microorganism that retains the ability to prevent the generation of foot malodor by skin microorganisms

34. An inactive form of the microorganism of claim 31, which is able to prevent the generation of foot malodor by skin microorganisms.

35. An inactive form of the microorganism of claim 32, which is able to prevent the generation of foot malodor by skin microorganisms.

36. The inactive form of claim 34, which is thermally inactivated or lyophilized.

37. A composition comprising the microorganism of claim 22.

38. A cosmetic composition, which comprises the composition of claim 37 and a cosmetically acceptable carrier or excipient.

39. A pharmaceutical composition, which comprises the composition of claim 37 and a pharmaceutically acceptable carrier or excipient.

40. A method for the production of a cosmetic composition comprising the step of formulating the microorganism of claim 22 with a cosmetically acceptable carrier or excipient.

41. A method for the production of a pharmaceutical composition comprising the step of formulating the microorganism of claim 22 with a pharmaceutically acceptable carrier or excipient.
